(19) United States
(12) Patent Application Publication
(10) Pub. No.: US 2011/0251229 A1
(43) Pub. Date: Oct. 13, 2011

(54) (+)-OPIOIDS AND METHODS OF USE

(75) Inventors: Linda May Rothblum Watkins, Boulder, CO (US); Mark Rowland Hutchinson, Glenalta (AU); Kenner C. Rice, Bethesda, MD (US)

(73) Assignee: The Regents of the University of Colorado, Denver, CO (US)

(21) Appl. No.: 12/740,688

(22) PCT Filed: Oct. 30, 2008
(86) PCT No.: PCT/US2008/081860

§ 371 Date: Apr. 27, 2011

Related U.S. Application Data
(60) Provisional application No. 60/983,927, filed on Oct. 30, 2007.

(51) Int. Cl. A61K 31/439; A61P 25/04

(52) U.S. Cl. 514/289

(57) ABSTRACT

The invention provides 4,5-epoxymorphinan or a derivative thereof, a morphinan or a derivative thereof, or a pharmaceutical salt or a prodrug thereof. The present invention also provides compositions comprising the same, and methods for using the same. In particular, the invention relates to TLR antagonistic opioids and methods for using the same.

### CROSS-REFERENCE TO RELATED APPLICATIONS

This application claims the priority benefit of U.S. Provisional Application No. 60/983,927, filed Oct. 30, 2007, which is incorporated herein by reference in its entirety.

### STATEMENT REGARDING FEDERALLY FUNDED RESEARCH

The U.S. Government has a paid-up license in this invention and the right in limited circumstances to require the patent owner to license others on reasonable terms as provided for by the terms of Grant Nos. DA015642, DA017670, DA024044, and DE017782 awarded by the National Institutes of Health.

### FIELD OF THE INVENTION

The invention relates to (+)-opioids or a derivative thereof, compositions comprising the same, and methods for using the same.

### BACKGROUND OF THE INVENTION

The pharmacology and treatment of pain has a very long and tumultuous history. Since the infancy of the use of opium poppy extracts to treat pain around 3500 BC, the search for treatments that provide effective relief from acute and chronic pain has continued to grow at an extraordinary rate. Today, pain still remains a significant public health issue with two-thirds of patients achieving little to no pain relief from the myriad of currently available pharmacotherapies and dosing regimens. The use of opioid pharmacotherapies produces several rewarding and reinforcing side effects, which result in the drugs' diversion to abuse settings. Unfortunately, a significant side effect in attempting to improve patients' quality of life is that some become dependent to the treatments that were prescribed to help them. In recent years the misuse of opioids has risen drastically, leaving doctors and patients hesitant to treat pain to the fullest extent.

Therefore, there is a continuing need for compounds, compositions, and methods for treating pain.

### SUMMARY OF THE INVENTION

The invention relates to a toll like receptor (TLR) modulator comprising a 4,5-epoxymorphinan or a derivative thereof, a morphinan or a derivative thereof, or a pharmaceutical salt or a prodrug thereof. In some embodiments, TLR modulators are TLR antagonists. In general, it has been found by the present inventors that (+)-isomers have TLR antagonistic activity.

In some embodiments of the invention, the TLR modulator, typically TLR antagonist, comprises a compound of the formula:

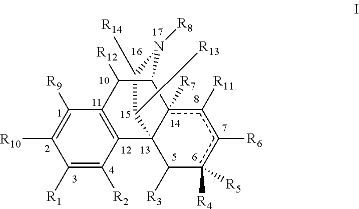

or a pharmaceutical salt thereof, compositions comprising the same, and methods for using the same.

Within compounds of Formula I:
each of R¹, R⁹, R¹⁰, R¹¹, R¹² is independently H, alkyl, —XR¹⁵, halide, —NR¹⁶R¹⁷, carboxyl, hydroxyalkyl, haloalkyl, cycloalkyl, (cycloalkyl)alkyl, acyl, alkoxyalkyl, alkoxycarbonyl, alkyl-carboxyl, aryl, aralkyl, aryloxy, arylthio, alkylthio, or amido;
R² is hydrogen, alkyl, halide, OR′, where R′ is hydrogen, hydroxyalkyl, haloalkyl, cycloalkyl, (cycloalkyl)alkyl, alkoxyalkyl, alkenyl, aryl, aralkyl, aryloxy, alkylthio, amino, amido, carboxyl, or —C(OR^{b})R^{c}R^{d}, wherein each of R^{b}, R^{c}, and R^{d} is independently H, alkyl, cycloalkyl, phenyl, or phenalkyl;
R³ is hydrogen, alkyl, or halide;
or R² and R³ together form —NR^{a}—, —O—, or —S—, where R^{a} is hydrogen, alkyl, or a nitrogen protection group;
R⁴ is H, —XR¹⁵, or —OC(═O)—R¹⁸;
R⁵ is H;
or R⁴ and R⁵ together form ═Y;
R⁶ is H, —XR¹⁹, alkyl, —C(═O)—R¹⁸, hydroxyalkyl, haloalkyl, cycloalkyl, (cycloalkyl)alkyl, alkoxyalkyl, alkenylalkyl, aryl, aralkyl, aryloxy, alkylthio, amino, amido, carboxyl, or —C(OR^{b})R^{c}R^{d}, wherein each of R^{b}, R^{c}, and R^{d} is independently H, alkyl, cycloalkyl, phenyl, or phenalkyl;
R⁷ is H, alkyl, or —XR¹⁵;
or R⁵ and R⁷ together form —(CH₂)ₙ—;
R⁸ is H, alkyl, haloalkyl, a nitrogen protecting group, (cycloalkyl)alkyl, or alkenyl; each of R¹³ and R¹⁴ is independently halide, oxime, alkyl, arylalkyl, CN, —OR¹⁸ and —OC(═O)—R¹⁸;
each R¹⁵ is independently H, alkyl, hydroxyalkyl, haloalkyl, alkoxyalkyl, haloacyl, acyl, aryl, aralkyl, or carbohydrate or a derivative thereof;
R¹⁶ is hydrogen, alkyl, or a nitrogen protecting group;
each of R¹⁷, R¹⁸, and R¹⁹ is independently hydrogen or alkyl;
n is an integer from 1 to 4;
each X is independently O or S;
Y is O or CH₂; and
dashed line indicates an optional double bond provided that when C₇-C₈ is a double bond, C₆-C₇ and C₈-C₁₄ are single bonds, and provided that when C₈-C₁₄ is a double bond R⁷ is absent.

Another aspects of the invention provide a method for modulating Toll-like receptor (TLR) comprising contacting a cell expressing a TLR with an effective amount of a TLR antagonist of a Compound of Formula I.

Still other aspects of the invention provide a composition comprising a Compound of Formula I and a Toll like receptor (TLR) agonist.

In some embodiments, the TLR agonist is a first opioid or a derivative thereof. Within these embodiments, in some instances the TLR antagonist is not an enantiomer of the first opioid or a derivative thereof. In other embodiments, the TLR antagonist is an enantiomer of the first opioid or a derivative thereof.

Yet other aspects of the invention provide a method for potentiating analgesic effects of an opioid on a subject. The method generally comprises administering to the subject who is on an opioid treatment a Compound of Formula I.

Within these aspects, in some embodiments, the Compound of Formula I is administered to the subject substantially simultaneously with the opioid.

Still other aspects of the invention provide a method for reducing the risk of developing an opioid dependency by a subject during an opioid pain relief therapy. The methods generally comprise administering to the subject who is on an opioid pain relief therapy a Compound of Formula I.

Other aspects of the invention provide a method for treating a subject for a clinical condition associated with Toll-like receptor (TLR) activation. The methods generally comprise administering to the subject a Compound of Formula I.

In some embodiments of the invention, the clinical condition comprises a condition associated with Toll-like receptor (TLR) mediated glial activation.

Still in other embodiments of the invention, the clinical condition comprises neuropathic pain, nociception, acute and repetitive opioid analgesia, rewarding effects of drug abuse, multiple sclerosis pain, spinal cord injury pain, headaches, cancer pain, as well as other forms of chronic pain, opioid dependency, or a combination thereof.

Yet other aspects of the invention provide a composition comprising an admixture of an analgesic opioid and a TLR antagonist comprising a 4,5-epoxymorphinan or a derivative thereof, a morphinan or a derivative thereof, or a pharmaceutical salt or a prodrug thereof.

### BRIEF DESCRIPTION OF THE DRAWINGS

FIG. 1 is a schematic illustration of neuropathic pain highlighting theoretical points (A through E) where some of the pharmacological targets can be designed to treat neuropathic pain to which glia contribute.

FIG. 2 is a graph showing that chronic constriction injury (CCI)-induced allodynia is reversed by acute blockade of TLR4.

FIG. 3 is a graph showing (+)-Naloxone non-competitively antagonizes activation of TLR4 by lipopolysaccharide (LPS).

FIG. 4 is a graph showing systemic (+)-naloxone acutely reverses chronic constriction injury-induced allodynia.

FIG. 5 is a graph showing inhibition of TIRAP signaling potentiates intrathecal morphine analgesia.

FIG. 6 is a graph showing potentiation of morphine analgesia by co-administration of LPS antagonist.

FIGS. 7A and 7B are graphs showing potentiation of morphine analgesia by (+)-naloxone and (+)-naltrexone, respectively.

FIG. 7C is a graph showing potentiation of morphine analgesia by (+)-naloxone delivered subcutaneously.

FIG. 8A shows assessment results of hyperalgesia and analgesia in rats treated with morphine and morphine+naloxone.

FIG. 8B shows withdrawal behaviors of rats in FIG. 8A that were treated with (−)-naltrexone to precipitate opioid withdrawal.

FIG. 9A shows the amount of TLR4 mRNA expression in the lumbar dorsal spinal cord of rats treated with various opioids.

FIG. 9B shows the amount of TLR4 mRNA expression in isolated microglial after treatment with morphine.

FIGS. 10A and 10B shows graphs of attenuation of both ipsilateral and contralateral, respectively, by naloxone.

FIG. 11A is a bar graph of quantitative integrated density following chronic intrathecal treatment with saline, (+)-naloxone or (−)-naloxone.

FIG. 11B is microphotographs of CD11b (OX42) staining in the left dorsal horn of rats with chronic constriction injury of the left sciatic nerve following chronic intrathecal treatment with saline, (+)-naloxone or (−)-naloxone, respectively.

FIG. 12 is a table of in vitro TLR agonism data for various compounds.

FIG. 13 is a table of in vitro TLR antagonism date for various representative compounds of the invention.

FIG. 14A is in silico TLR4 & TLR2 docking simulations of (−)-morphine.

FIG. 14B is in silico TLR4 docking simulations of (−)- and (+)-morphine.

FIG. 14C is in silico TLR4 docking simulation of (+)- and (−)-naloxone.

FIG. 14D is in silico TLR4 docking simulation of morphine-3-glucuronide (M3G) and morphine-6-glucuronide (M6G) the 2 main metabolites of morphine.

FIG. 14E is a graph of in silico docking energies (y-axis) vs. in vitro % reduction in LPS response (x-axis) of various compounds.

FIGS. 15A-H are graphs showing potentiation of morphine analgesia by co-administration with various compounds of the invention.

FIG. 16A is a graph showing a comparison experiment of the agonist (and lack of) activity of opioids on TLR4 transfected HEK293 cells compared to LPS.

FIG. 16B is a graph showing attenuation of morphine and M3G induced TLR4 signalling in the TLR4 transfected HEK293 cells by a variety of doses of (+)-naloxone.

FIGS. 17A and 17B show results of LPS-induce TLR4 signalling and morphine-induced Akt1 response in the presence of (+)-naloxone, respectively, using a RAW264.7 mouse macrophage cell line that stably expresses green fluorescent protein (GFP)-tagged Akt1.

FIGS. 18A-H show results of the L5 spinal cord of CCI rats receiving sustained intrathecal administration of 60 μg/hr (−)-naloxone, 60 μg/hr (+)-naloxone, or vehicle across 4 days that were analyzed for expression of astrocyte (GFAP; FIGS. 18A and B) and microglia (CD11b/c; FIGS. 18C-H) activation markers.

FIG. 19 shows graphs of attenuation of both ipsilateral and contralateral mechanical allodynia by systemic (+)-naloxone.

FIG. 20 shows the effects of (+)-naloxone on microglial activation by LPS.

### DETAILED DESCRIPTION OF THE INVENTION

### Definitions

“Acyl” refers to a moiety of the formula —C(═O)CH₃.

“Alkyl” refers to a saturated linear monovalent hydrocarbon moiety of one to twelve, preferably one to six, carbon atoms or a saturated branched monovalent hydrocarbon moiety of three to twelve, preferably three to six, carbon atoms. Exemplary alkyl group include, but are not limited to, methyl, ethyl, n-propyl, 2 propyl, tert-butyl, pentyl, and the like.

“Alkenyl” refers to a linear monovalent hydrocarbon moiety of two to ten carbon atoms or a branched monovalent hydrocarbon moiety of three to ten carbon atoms, containing at least one double bond, e.g., ethenyl, propenyl, and the like.

“Alkylene” refers to a saturated linear saturated divalent hydrocarbon moiety of one to twelve, preferably one to six, carbon atoms or a branched saturated divalent hydrocarbon moiety of three to twelve, preferably three to six, carbon atoms. Exemplary alkylene groups include, but are not limited to, methylene, ethylene, propylene, butylene, pentylene, and the like.

“Alkoxy” refers to a moiety of the formula —ORⁿ, where Rⁿ is alkyl as defined herein.

“Alkoxyalkyl” refers to a moiety of the formula —R^{p}—O—R^{q}, where R^{p} is alkylene and R^{q} is alkyl as defined herein.

“Alkoxycarbonyl” refers to a moiety of the formula —C(═O)—ORⁿ, where Rⁿ is alkyl as defined herein.

“Alkyl-carboxyl” refers to a moiety of the formula —C(═O)—Rⁿ, where Rⁿ is alkyl as defined herein.

“Alkylthio” refers to a moiety of the formula —SRⁿ, where Rⁿ is alkyl as defined herein.

“Amido” refers to a moiety of the formula —NR^{e}R^{f}, where each of R^{e} and R^{f} is independently H or alkyl as defined herein.

“Antagonist” refers to a compound or a composition that attenuates the effect of an agonist. The antagonist can bind reversibly or irreversibly to a region of the receptor in common with an agonist. Antagonist can also bind at a different site on the receptor or an associated ion channel. Moreover, the term “antagonist” also includes functional antagonist or physiological antagonist. Functional antagonist refers to a compound and/or compositions that reverses the effects of an agonist rather than acting at the same receptor, i.e., functional antagonist causes a response in the tissue or animal which opposes the action of an agonist. Examples include agents which have opposing effects on an intracellular second messenger, or, in an animal, on blood pressure. A functional antagonist can sometimes produce responses which closely mimic those of the pharmacological kind.

“Aryl” refers to a monovalent mono-, bi- or tricyclic aromatic hydrocarbon moiety of 6 to 15 ring atoms which is optionally substituted with one or more, preferably one, two, or three substituents within the ring structure. When two or more substituents are present in an aryl group, each substituent is independently selected.

“Aryloxy” and “arylthio” refer to a moiety of the formula —Z—Ar¹, where Ar¹ is aryl as defined herein and Z is O and S, respectively.

“Aralkyl” refers to a moiety of the formula —R^{x}R^{y} where R^{x} is an alkylene group and R^{y} is an aryl group as defined herein. Exemplary aralkyl groups include, but are not limited to, benzyl, phenylethyl, 3-(3-chlorophenyl)-2-methylpentyl, and the like.

“Carboxyl” refers to a moiety of the formula —C(═O)—R^{m}, where R^{m} is alkyl, aryl, or aralkyl.

“Chiral center” (i.e., stereochemical center, stereocenter, or stereogenic center) refers to an asymmetrically substituted atom, e.g., a carbon atom to which four different groups are attached. The ultimate criterion of a chiral center, however, is nonsuperimposability of its minor image.

“Cycloalkyl” refers to a non-aromatic, preferably saturated, monovalent mono- or bicyclic hydrocarbon moiety of three to ten ring carbons. The cycloalkyl can be optionally substituted with one or more, preferably one, two, or three, substituents within the ring structure. When two or more substituents are present in a cycloalkyl group, each substituent is independently selected.

“(Cycloalkyl)alkyl” refers to a moiety of the formula —R^{v}R^{w} where R^{v} is an alkylene group and R^{w} is a cycloalkyl group as defined herein. Exemplary cycloalkylalkyl groups include, but are not limited to, cyclopropylmethyl, cyclohexylpropyl, 3-cyclohexyl-2-methylpropyl, and the like.

The terms “halo,” “halogen” and “halide” are used interchangeably herein and refer to fluoro, chloro, bromo, or iodo.

“Haloalkyl” refers to an alkyl group as defined herein in which one or more hydrogen atom is replaced by same or different halo atoms. The term “haloalkyl” also includes perhalogenated alkyl groups in which all alkyl hydrogen atoms are replaced by halogen atoms. Exemplary haloalkyl groups include, but are not limited to, —CH₂Cl, —CF₃, —CH₂CF₃, —CH₂CCl₃, and the like.

“Heterocyclyl” means a non-aromatic monocyclic moiety of three to eight ring atoms in which one or two ring atoms are heteroatoms selected from N, O, or S(O)n (where n is an integer from 0 to 2), the remaining ring atoms being C, where one or two C atoms can optionally be a carbonyl group. The heterocyclyl ring can be optionally substituted independently with one or more, preferably one, two, or three, substituents. When two or more substituents are present in a heterocyclyl group, each substituent is independently selected. Exemplary heteorcyclyl groups include, but is not limited to, tetrahydropyranyl, piperidino, piperazino, morpholino and thiomorpholino, thiomorpholino-1-oxide, thiomorpholino-1,1-dioxide, and the derivatives thereof.

“Hydroxyalkyl” refers to an alkyl group having one or more hydroxyl substituent.

“Enantiomeric excess” refers to the difference between the amount of enantiomers. The percentage of enantiomeric excess (% ee) can be calculated by subtracting the percentage of one enantiomer from the percentage of the other enantiomer. For example, if the % ee of (R)-enantiomer is 99% and % ee of (S)-enantiomer is 1%, the % ee of (R)-isomer is 99%-1% or 98%.

“Leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N,O-dimethylhydroxylamino, and the like.

“Pharmaceutically acceptable excipient” refers to an excipient that is useful in preparing a pharmaceutical composition that is generally safe, non-toxic and neither biologically nor otherwise undesirable, and includes excipient that is acceptable for veterinary use as well as human pharmaceutical use.

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The terms “pro-drug” and “prodrug” are used interchangeably herein and refer to any compound which releases an active parent drug according to Formula I in vivo when such prodrug is administered to a mammalian subject. Prodrugs of a compound of Formula I are prepared by modifying one or more functional group(s) present in the compound of Formula I in such a way that the modification(s) may be cleaved in vivo to release the parent compound. Prodrugs include compounds of Formula I wherein a hydroxy, amino, or sulfhydryl group in a compound of Formula I is bonded to any group that may be cleaved in vivo to regenerate the free hydroxyl, amino, or sulfhydryl group, respectively. Examples of prodrugs include, but are not limited to, esters (e.g., acetate, formate, and benzoate derivatives), carbamates (e.g., N,N-dimethylaminocarbonyl) of hydroxy functional groups in compounds of Formula I, and the like.

“Protecting group” refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3rd edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

“Corresponding protecting group” means an appropriate protecting group corresponding to the heteroatom (i.e., N, O, P or S) to which it is attached.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

When describing a chemical reaction, the terms “treating”, “contacting” and “reacting” are used interchangeably herein, and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

As used herein, the terms “those defined above” and “those defined herein” when referring to a variable incorporates by reference the broad definition of the variable as well as any narrow definitions, if any.

The term “a derivative or an analog thereof” refers to those compounds that are derived from or having a similar core structure and retain all of the biological activity of the compound to which they are referred to. The term “all of the biological activity” refers to biological activities referred to herein when discussing the compound, e.g., TLR antagonistic property, etc.

“Chronic pain” refers to pain that persists longer than the temporal course of natural healing, associated with a particular type of injury or disease process.

“Nociceptive pain” refers to pain associated with the neuronal sensing and signaling resulting from potential injury or actual damage. Nociceptive pain is caused by an injury or disease outside the nervous system. It is often an on-going dull ache or pressure, rather than the sharper, trauma-like pain more characteristic of neuropathic pain. Nociceptive fibers signal tissue irritation, impending injury, or actual injury. When activated, they transmit pain signals (via the peripheral nerves as well as the spinal cord) to the brain. The pain is typically well localized, constant, and often with an aching or throbbing quality. Visceral pain is the subtype of nociceptive pain that involves the internal organs. It tends to be episodic and poorly localized. Nociceptive pain is usually time limited, e.g., when the tissue damage heals, the pain typically resolves. (Arthritis is a notable exception in that it is not time limited.) Typically, nociceptive pain tends to respond well to treatment with opioids. Exemplary nociceptive pains include sprains, bone fractures, burns, bumps, bruises, inflammation (from an infection or arthritic disorder), obstructions, and myofascial pain (which may indicate abnormal muscle stresses).

### Overview

Owing to the pain transmission capacity, neurons have been the primary intentional target of all pharmacotherapies developed to date. Generally, it is believed that opioids modulate pain solely by acting at neuronal opioid receptors and that opioid antagonists likewise exert their effects solely on neurons. Furthermore, it is conventionally believed that the detrimental (e.g., tolerance, hyperalgesia, dependence, and reward, etc.) and beneficial (e.g., analgesia, cough suppressant, etc.) actions of opioids are mediated via very similar and potentially inseparable mechanisms, reliant on neuronal opioid receptors.

In contrast, the present inventors have shown that the immunocompetent cells of the central nervous system (glia), their receptors, and their secreted signaling factors are involved in pain processing and opioid pharmacodynamics. In particular, glia have been shown to have a role in initiating and maintaining increased nociception in response to peripheral nerve injury. Recently, it has been suggested that glia can also modulate the analgesic actions of chronically administered opioids. Accordingly, some aspects of the invention provide pharmacological targeting (e.g., modulation) of glia to modulate (e.g., reduce or eliminate) pain and enhanced efficacy of opioids.

The present inventors also have shown that opioids cause direct glial activation in a non-classical opioid receptor fashion, via opioid-induced activation of a class of pattern recognition receptors termed Toll-like Receptors (TLRs). TLRs are significant mediators of neuropathic pain, opioid tolerance, opioid dependence, and opioid reward. Thus, in some instances antagonizing TLRs reverses neuropathic pain, and potentiates opioid and non-opioid analgesia. Also disclosed herein are the beneficial (e.g., classical neuronal opioid receptor mediated analgesia) and detrimental (e.g., glially mediated side effects) actions of analgesic compounds, such as opioids, and methods for modulating such.

As disclosed herein, glial activation also contributes significantly to neuropathic pain and to the development of opioid tolerance, opioid dependence and opioid reward. Thus, attenuation of glial activation alleviates neuropathic pain and reduces the development of opioid tolerance, dependence and reward. It is believed that opioid-induced glial activation occurs via a non-opioid receptor due to non-stereoselective agonist activity. Accordingly, some aspect of the invention relates to attenuating glial activation by antagonizing or blocking TLR (e.g., TLR2, TLR4, other TLR that can bind to either opioid analgesics, non-opioid analgesics or endogenous danger signals known to be TLR agonists, or a combination thereof) or generally reducing glial activation. Reduction of glial activation reduces exaggerated pain states, enhances opioid analgesia, and reduces the development of opioid tolerance, dependence and reward.

Some of the other clinical conditions associated with TLR include, but are not limited to, gastrointestinal pathologies (e.g., colitis, inflammatory bowel disease, Crohn's disease, irritable bowel disease, and celiac disease), cardiovascular disease (e.g., inflammatory heart disease, vascular inflammation, myocardial ischemia/reperfusion injury, and atherosclerosis), diabetes [e.g., diabetes/insulin resistance, (killing of islet cells)], immune related conditions (e.g., allergy, asthma, eczema, auto-immune disorders including arthritis, lupus and glomerulonephritis), systemic pathologies (e.g., primary or secondary sepsis, transplant organ rejection, and liver toxicity), neurodegeneration (e.g., neurodegenerative disorders generally, including Alzheimer's, Parkinson's, dementia, Multiple Sclerosis, Huntington's disease, Amyotrophic lateral sclerosis, and aging), and other physiological function (e.g., induction of labor, fever, seizures, epilepsy, and epileptogenesis). Accordingly, some aspects of the invention provide methods for treating a clinical condition associated with agonism of TLR.

Conventionally, glia (astrocytes and microglia) were viewed as structural supports for neurons and important for maintaining central nervous system (CNS) homeostasis. Glia were long overlooked in pain research due to their lack of axons and their yet-to-be-discovered roles in cell-to-cell communication. The roles of CNS glia in providing immune surveillance, clearance of debris, and regulation of ionic and chemical composition of the extracellular space in the survival of the host are well known. However, a possible involvement of glia under varying pain states has only recently been investigated. One possible indication for a potential role of glia in pain regulation was an associative link between astrocyte activation and neuropathic pain, for example, drugs that blocked neuropathic pain also decreased glial activation.

Upon activation, the functions of microglia and astrocytes change in that they begin producing and releasing a variety of neuroexcitatory substances including traditional nociceptive modulators, such as reactive oxygen species, nitric oxide, prostaglandins, excitatory amino acids, growth factors, and proinflammatory cytokines, which was recently recognized. Principal among proinflammatory cytokines are interleukin (IL)-1, IL-6 and tumor necrosis factor-a. Without being bound by any theory, it is believed that spinal cord glia are one of the principal producers of these proinflammatory cytokines in the central nervous system. In fact, spinal glial activation and subsequent release of proinflammatory mediators are believed to be involved in initiating and maintaining diverse enhanced pain states including neuropathic pain.

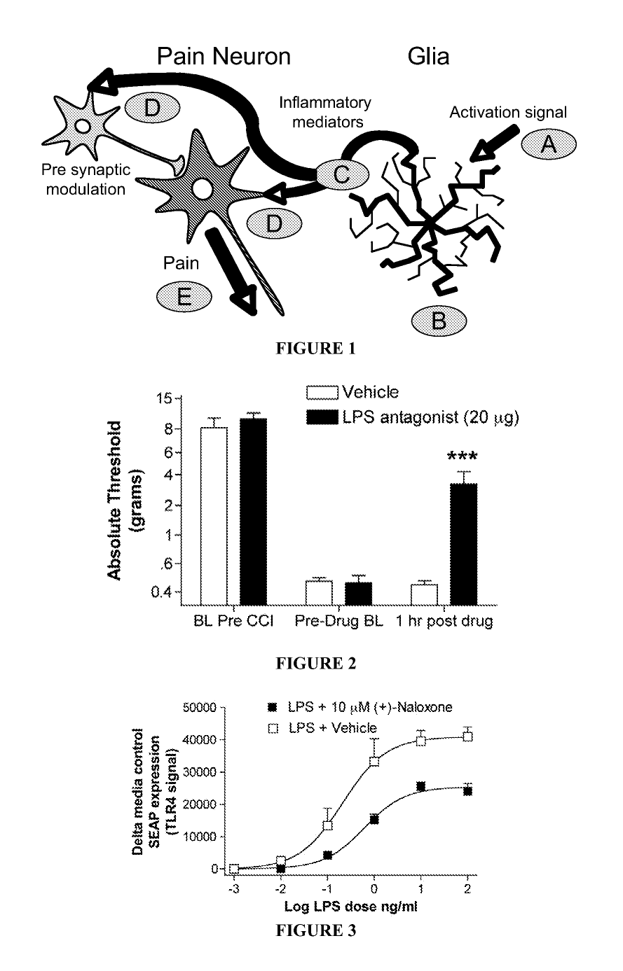

As shown in FIG. 1, there are numerous points along glial regulation of neuropathic pain where glia can be targeted to treat neuropathic pain. Traditional pain therapies have typically targeted transmission of the pain signal via neurons (step E; FIG. 1) with limited success. As can be seen in this schematic pathway, merely treating the neuronal component of the pathology leaves the glial component unabated, still attempting to communicate to neurons to propagate pain signals. It is possible glia are activating neurons via different pathways/intracellular signaling cascades than modulated by drugs targeting neurons. Perhaps this explanation may elucidate the unfortunate lack of generalized success of current pain therapies.

One of the initial steps in the neuropathic pain pathway is believed to be activation of glia (Step A in FIG. 1). A variety of glial activation signals have been identified. Signal(s) that initiates glial activation can vary depending on the insult delivered. Several mediators of glial activation are well characterized including neuronally-released fractalkine and traditional neuronal nociceptive modulators and transmitters, such as reactive oxygen species, nitric oxide, prostaglandins, excitatory amino acids, substance P, ATP, growth factors, and proinflammatory cytokines. In the majority of these cases, known receptor-mediated events have been characterized.

As can be seen in FIG. 1, a variety of points in neuropathic pain (A through E) can be targeted to treat neuropathic pain to which glia contribute. An activation signal or series of activation signals are required to activate glia (Step A in FIG. 1). Activation of glia is often mediated via cell surface receptors that can be antagonized. The term “glial activation” refers to the state in which glia release proinflammatory mediators. This state (i.e., glial activation, Step B in FIG. 1) can be modulated or attenuated thereby inhibiting various cellular events that block glial activation or its downstream consequences. An anti-inflammatory environment can also be produced which increases the threshold that an activation signal has to overcome to activate the cells.

Immune inflammatory mediators such as proinflammatory cytokines can be neutralized prior to reaching their intended receptor target (pre and/or post synaptic) by using soluble receptors (which exist endogenously), neutralizing antibodies, or compounds that decrease maturation of cytokines into their active form or increase the rate of cytokine degradation (Step C in FIG. 1). The action of many glial inflammatory mediators on neurons (pre and/or post synaptic) can also be antagonized at neuronal receptor sites (Step D in FIG. 1). There are myriads of currently employed neuronally targeted therapies that decrease the neuronal signaling of pain signals (pre and/or post synaptic, Step E in FIG. 1).

Some aspects of the invention relate to modulating initiator and mediator of neuropathic pain that involve signals relayed by Toll-like Receptors (TLRs), such as TLR2, TLR4, other TLR that recognizes endogenous danger signals, or a combination thereof. TLRs are a family of approximately 10 single transmembrane receptors that recognize a diverse range of moieties or “patterns” on exogenous (e.g., lipopolysaccharide [LPS] of gram-negative bacteria such as E. coli and Salmonella) and endogenous (e.g., heat shock proteins and cell membrane components released from damaged cells) substances that are considered to be danger signals and hence warrant activation of the innate immune system aimed at defending the survival of the host. TLR4 has been extensively characterized, as it is the TLR that recognizes LPS. Binding of agonists to TLRs activate downstream intracellular signaling pathways (similar to IL-1 binding to its cognate receptor) resulting in a proinflammatory signal.

Some aspects of the invention modulate TLR2, TLR4, other TLR that can bind to either opioid analgesics, non-opioid analgesics or endogenous danger signals known to be TLR agonists, or a combination thereof. As disclosed herein, a wide variety of chemically diverse compounds can modulate TLR2, TLR4, other TLR as above, or a combination thereof. Without being bound by any theory, using TLR2 and TLR4 as exemplars, TLR2 and TLR4 are believed to be some (but not all) of the key TLRs for recognizing and responding to endogenous danger signals that are released by damaged, dying and dead neurons and other cells (host DNA and RNA, heat shock proteins, cell membrane components, etc) and more general aspects of tissue injury (plasma proteins, extracellular matrix degradation products, etc). The present inventors have shown that acute intrathecal administration of a selective TLR4 antagonist in normal rats suppresses well-established neuropathic pain induced by chronic constriction injury (FIG. 2).

FIG. 2 is a bar graph showing that when adult rats that were administered intrathecally with 20 μg of TLR4 antagonist (a mutant LPS from msbB mutant E. coli that binds to but does not activate TLR4; Invivogen, San Diego, Calif., USA), it acutely reverses allodynia induced by chronic constriction injury of the sciatic nerve (n=4 per group). This TLR4 antagonist is a mutant form of LPS which lacks the myristoyl fatty acid moiety of the lipid A and displays 1,000 to 10,000 fold reduction in activation of NF-κB. In FIG. 2, BL (baseline) pre CCI Von Frey testing of thresholds occurred the day of CCI surgery of the left sciatic nerve. Allodynia developed across the following 10 days with maximal allodynia achieved and maintained from this point for a further 21 days. Pre-drug baseline (BL) thresholds were assessed the morning of the test day. Acute intrathecal delivery via the lumbar approach of vehicle and drug was conducted under brief isoflurane anesthesia. One hour post drug Von Frey testing was conducted. All testing was conducted blinded to treatment group by two separate experimenters.

These data demonstrate ongoing stimulation of TLRs, which is likely caused by injury-induced endogenous danger signals. Peripheral nerve injury leads to protracted expression of heat shock proteins in proximal axons of damaged sensory neurons and degradation of presynaptic terminals. Nerve degeneration in the central nervous system occurs remarkably slowly, taking months to years. Therefore, it is clear that endogenous danger signals created as a result of nerve injury could produce perseverative activation of at least TLR2 and TLR4 and, thereby, a perseverative drive for maintaining neuropathic pain. Without being bound by any theory, it is believed that a parallel activation of at least TLR2 and TLR4 would be anticipated to occur in, and be causal to, spinal cord injury pain, post-stroke pain, multiple sclerosis pain and other pains of central nervous system origin. Accordingly, modulation of glial activation (Step A of FIG. 1) can be used to treat neuropathic pain.

Some aspects of the invention provide compounds and compositions that can modulate (e.g., antagonize) TLRs for neuropathic pain control. Given that TLR2, TLR4, and other TLRs can signal the presence of endogenous danger signals, some embodiments of the invention provide compounds and compositions that modulates TLR2, TLR4, other TLRs, or a combination thereof. In some embodiments, compounds and compositions of the invention are permeable to the blood-brain barrier.

The classical opioid receptor bind (−)-isomers of opioids selectively. The present inventors have conducted study to determine if some (+)-opioids (e.g., (+)-naloxone) could block LPS-induced activation of TLR4. Using a TLR4 stably transfected cell line (Invivogen) with a stable co-transfection of an NF-κB reporter gene (secreted embryonic alkaline phosphatase; SEAP) the present inventors have found a significant non-competitive antagonism of LPS activity at TLR4 by (+)-naloxone. See FIG. 3. In FIG. 3, a TLR4 (cloned human receptor) stably transfected cell line was incubated for 24 hours with LPS, with or without 10 μM (+)-naloxone. SEAP expression was quantified as an indirect measure of TLR4 activation. LPS alone produced an EC₅₀ of 0.21 ng/ml with an Eₘₐₓ of 40,956 (arbitrary fluorescence units, change from media and (+)-naloxone alone controls). (+)-Naloxone (10 μM) produced a significant 3 fold increase in the EC₅₀ and a 40% reduction in the Eₘₐₓ, while having no substantial effect on basal unstimulated SEAP expression and therefore no significant effect on basal NF-κB activation. This result indicates that the antagonism is non-competitive owing to the reduction in Eₘₐₓ.

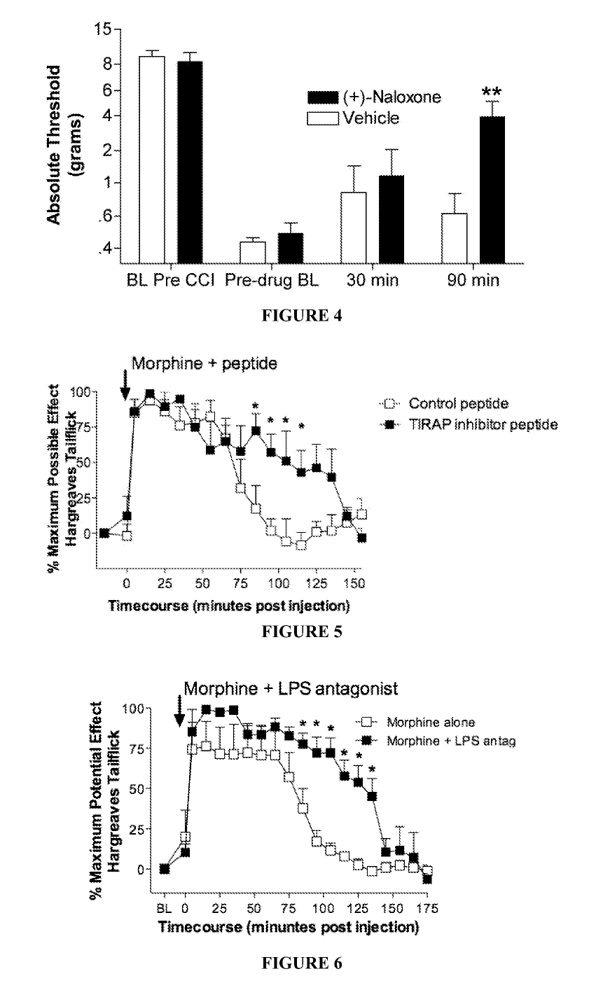

As shown in FIG. 4, (+)-naloxone also reversed CCI-induced allodynia 90 min following a systemic administration (60 mg/kg s.c.). These (+)-naloxone data indicate that blood brain barrier permeable small molecules can be used to antagonize TLR4 activity in vivo. In addition, TLR4 antagonism by small molecules can successfully reverse CCI-induced allodynia (Step A of FIG. 1). These data also show a role of TLR4 receptors in neuropathic pain. It is believed that opioid analgesia would be unaffected owing to the lack of opioid activity of the (+)-isomer. Other (+)-opioid isomers also show similar properties. For example, (+)-naltrexone delivery intrathecally or systemically reversed established CCI-induced allodynia, as did (−)-naltrexone. It appears naltrexone reverses neuropathic pain by non-stereoselectively antagonizing TLR4 receptors, acting in a non-classical opioid fashion similar to naloxone.

As shown in FIG. 4, (+)-naloxone (60 mg/kg s.c.) reversed established allodynia 90 min but not significantly in 30 min following drug administration (n=5 per group). BL (baseline) pre CCI Von Frey testing of thresholds occurred the day of CCI surgery of the left sciatic nerve. Allodynia developed across the following 10 days with maximal allodynia achieved and maintain from this point for a further 21 days. Pre-drug baseline (BL) thresholds were assessed the morning of the test day. All Von Frey testing was conducted blinded to treatment group by two separate experimenters 30 and 90 min post drug administration.

The present inventors have shown that some opioids, such as (−)-naloxone and (+)-naloxone, reverses neuropathic pain. Without being bound by any theory, it is believed that this activity is achieved via its actions as a TLR4 antagonist.

The mode of glial activation that results in enhanced pain can vary depending on the insult delivered. Thus, an effective treatment for neuropathic pain typically depends on which glial activating signal(s) are responsible for the pain pathway. A broader therapeutic approach is to inhibit or attenuate existing glial activation and/or products released by activated glia (e.g., Step D in FIG. 1). In some instances, compounds of the invention reverse neuropathic pain and return the animal toward normal basal pain responsivity, rather than producing analgesia. Therefore, all of these treatments are anti-allodynic and/or anti-hyperalgesic, leaving basal nociception unaffected.

As discussed previously, the inflammatory and pro-nociceptive mediators released by glia in their activated state are numerous. Therefore, clinically antagonizing or neutralizing each mediator (targeting steps C and/or D of FIG. 1) has its limitations as inhibiting the actions of each of these numerous mediators individually may be too great a task. However, in some instances proinflammatory cytokines appear to be one of the key factors in glial enhancement of pain. In some cases, neutralizing the action of principal proinflammatory cytokines (IL-1, IL-6, tumor necrosis factor-a, for example, Step C in FIG. 1) or antagonizing their receptors (e.g., Step D in FIG. 1) has proven a successful strategy for preventing and reversing neuropathic pain.

It has been observed that there is a similarity between the glial activation observed in response to peripheral neuropathy and the glial activation following chronic opioid exposure. It has also been observed that opioid agonists activate TLR2, TLR4, other TLR, or a combination thereof and some compounds (e.g., opioid antagonists) non-stereoselectively block one or more of these receptors.

The present inventors have found that TLRs are responsible for both neuropathic pain and opioid-induced glial activation. Accordingly, some aspects of the invention provide methods for modulating neuropathic pain, opioid-induced glial activation, or a combination thereof by administering a TLR antagonist or a composition comprising the same. In some embodiments, the TLR antagonist does not significantly compromise the pain-suppressive effects of opioids agonists on neurons.

Since the discovery of morphine modulation of T cell function in 1979, a large amount of work has been focused on characterizing the influence that opioid exposure has on the functioning of the immune system in its traditional role of host defense. However, the impact that the activation status of immunocompetent cells has on opioid actions has only been recently studied. While modulation of peripheral immune cells function by opioids is important to understanding host defense, these cells are not as likely as glia to have a profound effect on opioid pharmacodynamics. The immunocompetent cells that mediate effects on opioid analgesia are typically the glia of the dorsal root ganglia, spinal cord and brain. Peripheral immune cells have been implicated in many TLR-mediated clinical diseases, such as Crohn's disease.

A causal link between opioid-induced glial activation and the development of opioid tolerance has recently been recognized. It is believed that following chronic morphine administration, tolerance and morphine-induced hyperalgesia are produced, at least in part, as a consequence of glial activation. One mechanism that has been proposed to account for such effects is via nitric oxide induced p38 MAPK activation, with downstream up regulation of proinflammatory cytokines. Interleukin-1, interleukin-6 and tumor necrosis factor, in turn, oppose morphine analgesia.

It is believed that morphine is acting not only at classical opioid receptors on nociceptive neurons (step E, FIG. 1) but also as a glial activation signal (step A, FIG. 1) producing the same, or at least a similar cascade of events that results in increased nociception. The sum of morphine's neuronal anti-nociceptive activity and its pro-nociceptive glial activation results in a net reduction in analgesia. Moreover, glial activation increases with prolonged opioid treatment and results in an increasing analgesic tolerance. Furthermore, opioid-induced glial activation contributes significantly to the atypical allodynia and hyperalgesia that results from chronic opioid administration. The present inventors have found that IL-1, as well as other proinflammatory cytokines and chemokines, opposes morphine analgesia within minutes after either systemic or intrathecal administration.

The present inventors have observed similarity between neuropathy- and opioid-induced glial activations by using agents that reverse nerve injury-induced allodynia so as to define whether these same agents modulate morphine analgesia as well. The present inventors have discovered that agents that oppose neuropathic pain either by suppressing glial activation or by neutralizing or antagonizing proinflammatory glial products also oppose glial attenuation of both acute and chronic morphine analgesia. The efficacy of morphine can be potentiated by targeting opioid-induced glial activation (step B, FIG. 1) or by neutralizing (step C, FIG. 1) or antagonizing (step D, FIG. 1) the action proinflammatory cytokines.

It is believed that the activation of glia is not mediated via a classical “neuronal-like” opioid receptor. Using TLR4 antagonists that possess no classical opioid receptor activity, the present inventors have discovered the involvement of this non-classical opioid receptor in glial activation, which causes significant glial activation, allodynia and hyperalgesia, as well as upregulation of proinflammatory cytokine mRNA, protein and release. Glia do express classical opioid receptors. However, it is believed that the immunomodulation resulting from opioid exposure is not mediated by these classical opioid receptors.

Some aspects of the invention provide methods for using TLR antagonists to potentiate (−)-opioid (e.g., morphine) analgesia, for example, by blocking (−)-opioid induced glial activation and consequent increase in anti-analgesic proinflammatory cytokines. In some embodiments, TLR antagonists significantly potentiated both acute and chronic (−)-opioid analgesia.

Without being bound by any theory, it is believed that (−)-opioids that are used in treating pain are agonists of TLR2, TLR4, other TLRs, or a combination thereof. For example, when several clinically employed (−)-opioids were tested, they were all found to be TLR4 agonists. These opioid TLR4 agonists included morphine, methadone, oxycodone, buprenorphine, fentanyl and pethadine/meperidine, amongst others.

By targeting opioid-induced activation of glial TLRs, the present inventors were able to reduce or prevent this undesirable aspect of glial activation from progressing past step A (FIG. 1) that contributes to opioid-induced tolerance, allodynia and hyperalgesia. The beneficial neuronally-induced opioid analgesia is unhindered by opioid-induced glial activation.

It is believed that at least TLR4 is responsible for initiating a component of opioid-induced glial activation that contributes significantly to the unwanted pro-nociceptive side effects of opioid administration. Accordingly, some aspects of the invention provide methods for reducing unwanted pro-nociceptive side effects of opioid administration by administering a TLR antagonist.

It has been observed that several non-selective immunosuppressive treatments ameliorate morphine withdrawal behaviors. In addition, glial involvement in pain enhancement during morphine withdrawal is blocked by IL-1 receptor antagonist or IL-10.

Co-administration of a TLR antagonist [e.g., AV411 (ibudilast)] with an escalating dependence regimen of morphine significantly reduced naloxone precipitated withdrawal behaviors. Moreover, there was a corresponding reduction in glial activation in brain nuclei associated with opioid action.

In another experiment, a TLR antagonist was found to protect against previously established dependence and spontaneous withdrawal, as reflected by suppression of withdrawal induced spontaneous activity levels and weight loss. These data show that opioid-induced glial activation is involved in the development of morphine dependence and precipitation of withdrawal behaviors. Accordingly, some aspects of the invention provide methods for reducing opioid dependence, opioid withdrawal behaviors, or a combination thereof by administering a TLR antagonist. For example, the present inventors have observed that co-administration of a TLR antagonist significantly reduced withdrawal behaviors and attenuated morphine-induced weight loss.

As stated above, TLRs mediate the reinforcing and addictive actions of morphine. As such other aspects of the invention provide methods for increasing the beneficial actions, reducing the undesired effects, or a combination thereof of opioids. Such aspects of the invention often target glial activation. For example, it was observed that co-administration of a TLR antagonist resulted in a significant reduction in morphine reward.

Without being bound by any theory, it is believed that TLR-dependent glial activation results in neuropathic pain. Accordingly, some aspects of the invention provide methods for reducing neuropathic pain by modulating (e.g., reducing or preventing) TLR-dependent glial activation. One particular embodiment involves administering a TLR antagonist.

It is also believed that TLR-dependent opioid-induced glial activation results in undesired opioid side-effects, such as reducing opioid (e.g., morphine) analgesia, producing opioid dependence and reward, and causing respiratory depression. Therefore, other aspects of the invention provide methods for reducing or preventing TLR-mediated undesired opioid side-effects, for example, reduction in opioid analgesia, dependence, reward, or a combination thereof. “Undesired opioid side-effects” refers to effects of opioids other than analgesia. One particular embodiment involves administering a TLR antagonist.

The present inventors have also discovered that antagonizing TLRs or attenuating glial activation in neuropathic pain and during opioid exposure at least partially reverses allodynia and reduces unwanted opioid side effects, while maintaining opioid analgesic efficacy. The negative (i.e., undesired) side effects of opioids can be separated from the beneficial actions by, for example, targeting opioid-induced glial activation using blood brain barrier permeable pharmacotherapies such as TLR antagonists.

It is also believed that glial activation is at least partially responsible for the rewarding capacity of several abused compounds. Therefore, glial activation is a predictor for a patient's drug abuse liability. Examples of patient populations where this can pertain include HIV/AIDS, stress, and depression, etc. In all these cases, drug abuse is of considerable concern. Accordingly, some aspects of the invention provide methods for reducing or preventing drug abuse by administering a glial activation antagonist.

### Compounds

In some aspects, TLR antagonists of the invention comprise (+)-opioid compounds of the formula:

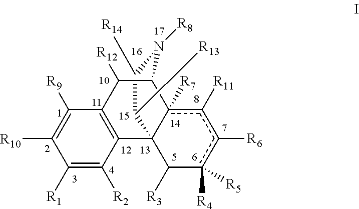

or a pharmaceutical salt thereof,
where
each of R¹, R⁹, R¹⁰, R¹¹, R¹² is independently H, alkyl, —XR¹⁵, halide, —NR¹⁶R¹⁷, carboxyl, hydroxyalkyl, haloalkyl, cycloalkyl, (cycloalkyl)alkyl, acyl, alkoxyalkyl, alkoxycarbonyl, alkyl-carboxyl, aryl, aralkyl, aryloxy, arylthio, alkylthio, or amido;
R² is hydrogen, alkyl, halide, OR′, where R′ is hydrogen, hydroxyalkyl, haloalkyl, cycloalkyl, (cycloalkyl)alkyl, alkoxyalkyl, alkenyl, aryl, aralkyl, aryloxy, alkylthio, amino, amido, carboxyl, or —C(OR^{b})R^{c}R^{d}, wherein each of R^{b}, R^{c}, and R^{d} is independently H, alkyl, cycloalkyl, phenyl, or phenalkyl;
R³ is hydrogen, alkyl, or halide;
or R² and R³ together form —NR^{a}—, —O—, or —S—, where R^{a} is hydrogen, alkyl, or a nitrogen protection group;
R⁴ is H, —XR¹⁵, or —OC(═O)—R¹⁸;
R⁵ is H;
or R⁴ and R⁵ together form ═Y;
R⁶ is H, —XR¹⁹, alkyl, —C(═O)—R¹⁸, hydroxyalkyl, haloalkyl, cycloalkyl, (cycloalkyl)alkyl, alkoxyalkyl, alkenylalkyl, aryl, aralkyl, aryloxy, alkylthio, amino, amido, carboxyl, or —C(OR^{b})R^{c}R^{d}, wherein each of R^{b}, R^{c}, and R^{d} is independently H, alkyl, cycloalkyl, phenyl, or phenalkyl;
R⁷ is H, alkyl, or —XR¹⁵;
or R⁵ and R⁷ together form —(CH₂)ₙ—;
R⁸ is H, alkyl, haloalkyl, a nitrogen protecting group, (cycloalkyl)alkyl, or alkenyl;
each of R¹³ and R¹⁴ is independently halide, oxime, alkyl, arylalkyl, CN, —OR¹⁸ and —OC(═O)—R¹⁸;
each R¹⁵ is independently H, alkyl, hydroxyalkyl, haloalkyl, alkoxyalkyl, haloacyl, acyl, aryl, aralkyl, or carbohydrate or a derivative thereof;
R¹⁶ is hydrogen, alkyl, or a nitrogen protecting group;
each of R¹⁷, R¹⁸, and R¹⁹ is independently hydrogen or alkyl;
n is an integer from 1 to 4;
each X is independently O or S;
Y is O or CH₂; and
dashed line indicates an optional double bond provided that when C₇-C₈ is a double bond, C₆-C₇ and C₈-C₁₄ are single bonds, and provided that when C₈-C₁₄ is a double bond R⁷ is absent.

In some embodiments of the invention, R¹ R is —OR¹⁵, wherein R¹⁵ is hydrogen, alkyl, or a carbohydrate or a derivative thereof.

Still in other embodiments of the invention, R⁴ is —OR¹⁵, or —OC(═O)—R¹⁹, and R⁴ is H, where R¹⁵ is hydrogen, alkyl, or a carbohydrate or a derivative thereof, and R¹⁹ is alkyl. Alternatively, R⁴ and R⁵ together form ═O or ═CH₂.

Yet in other embodiments of the invention, R⁷ is H, —OR¹⁵, and R¹⁵ is hydrogen or alkyl. Alternatively, R⁵ and R⁷ together form —(CH₂)₂—.

In other embodiments of the invention, R⁸ is alkyl, haloalkyl, (cycloalkyl)alkyl, or alkenyl.

Still in other embodiments of the invention, R⁶ is hydrogen or hydroxyalkyl.

In other embodiments of the invention, X is O.

Yet in other embodiments of the invention, C7-C8 is a double bond, and C6-C7 and C8-C14 are single bonds.

Still in other embodiment of the invention, C7-C8 is a single bond, and C6-C7 and C8-C14 are double bonds and R7 is absent.

Yet still in other embodiments, various R¹⁰ and R¹² substituents can be made, for example, by the procedures described in Tetrahedron Letters, 2003, 44(39), 7381-7384. In some instances, R¹⁰ is halide (e.g., Cl, Br, or I).

In other embodiments, various R¹¹ substituents can be made, for example, by the procedures described in Helvetica Chimica Acta, 2006, 89(5), 861-869. In some instances R¹¹ comprises O or N heteroatom substituent.

Still in other embodiments, various R¹³ or R¹⁴ substituents can be introduced, for example, by using the procedures described in Journal of the Chemical Society, Perkin Transactions 1: Organic and Bio-Organic Chemistry, (1972-1999) (1972), (6), 875-8 and Can. J. Chem., 1988, 66(10), 2498-2505. In some instances, R¹³ is halide (e.g., Cl, Br, or I), oxime, CN, —OH and —OAc. In other instances, R¹⁴ is alkyl or arylalkyl.

It should be recognized that combinations of various embodiments described herein form other embodiments. In this manner, a variety of compounds, compositions, and methods are embodied within the invention.

### Synthesis

Opium provides (−)-opioids, such as (−)-morphine, (−)-codeine and (−)-thebaine, that are useful in the manufacture of medical narcotics. Vast majority of opiate chemistry published to date (isolation of crystalline morphine from opium was first described in 1806) was developed using (−)-isomers of morphine, codeine and thebaine from opium poppies and their transformation products, or from sinomenine that has the carbon-nitrogen skeleton antipodal to the opium derived products. Those procedures derived from opium products are applicable to synthesis of the corresponding (+)-isomers since the required intermediates are now independently available either by total synthesis or from sinomenine (see below). Conversely, the procedures developed from the work with sinomenine are applicable to the synthesis of drugs with the natural opiate absolute configuration.

Some opiate synthesis and intermediates are described in U.S. Pat. Nos. 4,368,326, 4,410,700, 4,556,712, 4,521,601, 4,613,668, 5,008,449 and 5,668,285 and European patent EPO418591A2. Procedures disclosed in these patent are applicable to the synthesis of the unnatural (+)-enantiomers with absolute configuration shown in Formula 1 of the present application. These unnatural enantiomers generally have (+)-optical rotation although some exceptions are known such as simonenine, see below. Briefly, these procedures use commercially available starting materials such as 3-methoxyphenethylamine and 3-hydroxy-4-methoxyphenylacetic acid and provides either enantiomer of 1-bromo-nordihydrothebainone, dihydrothebainone, nordihydrocodeinone and dihydrocodeinone in about 40% overall yield. Either enantiomer of the entire spectrum of compounds described in the opiate chemistry prior art can be synthesized from the appropriate enantiomer of any one of the latter four compounds merely by applying published procedures.

Alternatively, (+)-dihydrocodeinone can be easily prepared from (−)-sinomenine, a commercially available plant alkaloid with the same absolute configuration as the unnatural opiates. The availability of (+)-dihydrocodeinone either by total synthesis or from (−)-sinomenine thus provides two independent routes to this intermediate and to the entire spectrum of unnatural opiates using procedures published for compounds in both the (−)-series and the (+)-series.

Accordingly, compounds of the invention can be readily prepared from available starting materials using the procedures known for (+)-opioids. Various substituents on the compounds of the invention can be present in the starting compounds, added to any one of the intermediates or added after formation of the final products by known methods of substitution or conversion reactions. For example, nitro groups can be added by nitration and the nitro group can be converted to other groups, such as amino by reduction, and halogen by diazotization of the amino group and replacement of the diazo group with halogen or simply by halogenation reaction. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

Additionally, as will be apparent to those skilled in the art, conventional protecting groups may be necessary to prevent certain functional groups from undergoing undesired reactions. The choice of a suitable protecting group for a particular functional group, as well as suitable conditions for protection and deprotection, are well known in the art. For example, numerous protecting groups, and their introduction and removal, are described in T. W. Greene and G. M. Wuts, Protecting Groups in Organic Synthesis, 3rd ed., John Wiley & Sons, New York, 1999, and references cited therein, all of which are incorporated herein by reference in their entirety.

Some of the methods for producing various (+)-opioid compounds are disclosed in European Patent Application No. 90116248.7 (Publication No. 0 418 591 A2). In addition, one skilled in the art can readily use methods described for synthesis of (−)-opioid compounds, for example, there are a number of U.S. Patents (such as those described above) that provide procedures on how to produce (−)-opiod compounds, to produce (+)-opioid compounds by using enantiomers. Optimum reaction conditions may vary with the particular reactants or solvent used, but such conditions can be determined by one skilled in the art by routine optimization procedures.

Since the compounds of the invention can have certain substituents which are necessarily present, the introduction of each substituent is, of course, dependent on the specific substituents involved and the chemistry necessary for their formation. Thus, consideration of how one substituent would be affected by a chemical reaction when forming a second substituent would involve techniques familiar to one of ordinary skill in the art. This would further be dependent on the ring involved.

In some instances, a racemic mixture of compounds of the invention can be prepared and the desired (+)-isomer can be resolved or separated (i.e., enantiomerically enriched) using any of the variety of chiral resolution methods known to one skilled in the art. Such resolution methods are described, for example, in the four volume compendium Optical Resolution Procedures for Chemical Compounds: Optical Resolution Information Center, Manhattan College, Riverdale, N.Y., and in Enantiomers, Racemates and Resolutions, Jean Jacques, Andre Collet and Samuel H. Wilen; John Wiley & Sons, Inc., New York, 1981, which are incorporated herein in their entirety.

In some resolution methods, a racemic mixture is converted to a mixture of diasteromers by attachment, either chemically or enzymatically, of a relatively enantiomerically pure moiety. Unlike enantiomers, most diastereomers have different physical properties, e.g., solubility, boiling point, affinity (e.g., to chromatography columns and enzymes), and the like. These different physical properties can be used to separate one diastereoisomer from another, for example, by fractional crystallization, distillation, chromatography, kinetic resolution using an enzyme, and the like.

Alternatively, the compound can be synthesized enantioselectively starting from enantiomerically pure or enriched starting material.

When the compound of the present invention contains an olefin moiety and such olefin moiety can be either cis- or trans-configuration, the compound can be synthesized to produce cis- or trans-olefin, selectively, as the predominant product. Alternatively, the compound containing an olefin moiety can be produced as a mixture of cis- and trans-olefins and separated using known procedures, for example, by chromatography as described in W. K. Chan, et al., J. Am. Chem. Soc., 1974, 96, 3642, which is incorporated herein in its entirety.

The compounds of the invention form salts with acids when a basic amino function is present and salts with bases when an acid function, e.g., carboxylic acid or phosphonic acid, is present. All such salts are useful in the isolation and/or purification of the new products. Of particular value are the pharmaceutically acceptable salts with both acids and bases. Suitable acids include, for example, hydrochloric, oxalic, sulfuric, nitric, benzenesulfonic, toluenesulfonic, acetic, maleic, tartaric and the like which are pharmaceutically acceptable. Basic salts for pharmaceutical use include Na, K, Ca and Mg salts.

### Pharmaceutical Compositions

The compounds of the invention can be administered to a patient to achieve a desired physiological effect. Typically the patient is a mammal, often human. The compound can be administered in a variety of forms adapted to the chosen route of administration, i.e., orally or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; rectal systemic, and central (e.g., intrathecal, such as into the cerebrospinal fluid around the spinal cord, and intracerebral into brain or CSF of the brain).

The active compound can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparation can contain at least 0.1% of active compound. The percentage of the compositions and preparation can, of course, be varied and can conveniently be between about 1 to about 10% of the weight of the unit. The amount of active compound in such therapeutically useful compositions is such that a suitable dosage will be obtained. Preferred compositions or preparations according to the present invention are prepared such that an oral dosage unit form contains from about 1 to about 1000 mg of active compound.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens a preservatives, a dye and flavoring such as cherry or orange flavor. Of course, any material used in preparing any dosage unit form should be pharmaceutically pure and substantially non-toxic in the amounts employed. In addition, the active compound can be incorporated into sustained-release preparations and formulation.

The active compound can also be administered parenterally. Solutions of the active compound as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the active compound in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The therapeutic compounds of the invention can be administered to a mammal alone or in combination with pharmaceutically acceptable carriers, as noted above, the proportion of which is determined by the solubility and chemical nature of the compound, chosen route of administration and standard pharmaceutical practice.

The physician will determine the dosage of the present therapeutic agents which will be most suitable for prophylaxis or treatment and it will vary with the form of administration and the particular compound chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

Additional objects, advantages, and novel features of this invention will become apparent to those skilled in the art upon examination of the following examples thereof, which are not intended to be limiting.

### EXAMPLES

### Synthesis

(+)-Morphine can be synthesized following the procedures described in, for example, Science, 1977, 198, 842-845; Heterocycles, 1977, 6, 1157-1165; and J. Org. Chem., 1978, 43, 1462-1463.

(−)-N-Cyclopropylmethylnordihydrocodeinone can be synthesized by alkylation of (−)-nordihydrocodeinone with cyclopropylmethylbromide using the method described in, for example, U.S. Pat. No. 2,741,613.

(−)-Nordihydrocodeinone can be also prepared using the method described in U.S. Pat. No. 2,741,613. It can also be synthesized from naltrexone, see for example, Heterocycles, 2006, 69, 271-282.

(+)-N-Cyclopropylmethylnordihydrocodeinone can be prepared by alkylation of (+)-nordihydrocodeinone with cyclopropylmethylbromide using the method described in U.S. Pat. No. 2,741,613 for the (−)-enantiomer. (+)-Nordihydrocodeinone can be obtained by the methods described in U.S. Pat. No. 4,521,601.

(+)-Dextrorphan, (−)-levorphanol, (+)-dextrallorphan and levallorphan can be prepared using methods described in, for example, Helv. Chim. Acta, 1951, 34, 2211.

(+)-Thevinone and (+)-19-Propylthevinol can be prepared following the procedure described in, for example, EP0418591A2 published Mar. 27, 1991.

Dextromethorphan was purchased from Sigma-Aldrich Chemical Co.

(+)-Naloxone can be prepared following the procedure described in, for example, J. Med. Chem., 1978, 21, 398-400.

(+)-Naloxone, (+)-naltrexone and (+)-nalmefene can also be prepared following the procedure described in, for example, U.S. Pat. No. 5,668,285.

C-1 substituents on opioid structure can be introduced. For example, 1-bromo and 1-carbethoxy substituents can be introduced following the procedure described in, for example, J. Org. Chem., 2005, 70, 6492-6495. 1-Methyl substituent can be introduced using procedures described in, for example, Rec. Tray. Chim. des Pays-Bas, 1976, 95, 189.

C-4 substituents can be introduced using procedures described in, for example, U.S. Pat. Nos. 4,368,326 and 4,521,601.

C-5 substituents can be introduced using procedures described in, for example, Helv. Chim. Acta, 1988, 71, 642.

C-6 keto substituents can be introduced using procedures described in, for example, U.S. Pat. Nos. 4,368,326 and 4,521,601. C-6 amino substituents can be introduced using procedures described in, for example, J. Med. Chem., 1981, 24, 903.

C-7 substituent can be introduced using procedures described in, for example, J. Org. Chem., 1995, 60, 2276.

C-14 alkyl can be introduced using procedures described in, for example, Helv. Chim. Acta, 1990, 73, 326. C-14 OH can be introduced using procedures described in, for example, J. Med. Chem., 1978, 21, 398-400 and U.S. Pat. No. 5,668,285.

### Methods and Materials

### General Methods

### Subjects

Pathogen-free adult male Sprague-Dawley rats (300-375 g; Harlan Labs, Madison, Wis.) were used in all experiments. Rats were housed in temperature (23±3° C.) and light (12 h:12 h light:dark cycle; lights on at 0700) controlled rooms with standard rodent chow and water available ad libitum. Each study involves n=approximately 6 per group.

### Catheter Implantation

The method of constructing and implanting the indwelling intrathecal catheters in rats was based on that described previously (J. Neurosci. Methods, 1999 90 81-86) Briefly, intrathecal catheters were implanted under anesthesia (isoflurane; Phoenix Pharmaceuticals, St. Joseph, Mo., USA) by threading sterile polyethylene-10 tubing (PE-10 Intramedic Tubing; Becton Dickinson Primary Care Diagnostics, Sparks, Md., USA) guided by an 18-gauge needle between the L5 and L6 vertebrae. The catheter was inserted 8.8 cm beyond the exterior end of the needle such that the proximal catheter tip lay over the lumbosacral enlargement. The needle was removed and the catheter was sutured to the superficial musculature of the lower back.

Catheter method 1: For acute intrathecal drug experiments the exterior end of the catheter was led subcutaneously to exit through a small incision at the nape of the neck. These catheters were preloaded with drugs at the distal end in a total volume of no greater than 25 μl. The catheters were 90 cm in length, allowing remote drug delivery without touching or otherwise disturbing the rats during the testing.

Catheter method 2: Chronic intrathecal indwelling catheters were prepared as above, but had an osmotic minipump attached to the end of the catheter. These catheters were 20 cm in total length.

Catheter method 3: For the subcutaneous catheter, a 90 cm length of PE-10 tubing was sutured to the superficial musculature of the lower back at the same time as the intrathecal catheter was implanted in these animals. The exterior end of the subcutaneous catheter paralleled the intrathecal catheter out of the same incision in the nape of the neck, allowing for remote subcutaneous administration without disturbance of the animals.

### Behavioral Measures

### Hargreaves Tests for Analgesia and Hyperalgesia

Rats received at least three 60 min habituations to the test environment prior to behavioral testing. Latencies for behavioral response to heat stimuli applied to the plantar surface of each hind-paw and tail were assessed using a modified Hargreaves test. All testing was conducted blind with respect to group assignment. Pilot studies determined that intrathecal catheter surgery did not affect baseline responses after 2 h or 7 days recovery from surgery, compared to latencies recorded prior to surgery. Briefly, baseline withdrawal values were calculated from an average of 2 consecutive withdrawal latencies of the tail and the left and the right hind-paws, measured at 15-min intervals. Latencies for the short baseline latency Hargreaves stimuli at baseline ranged from 3 to 4 s, and a cut-off time of 10 s was imposed to avoid tissue damage. Latencies for the long baseline latency Hargreaves stimuli at baseline ranged from 8 to 10 s, and a cut-off time of 20 s was imposed to avoid tissue damage. Short and long baseline latency stimuli were both employed to enable quantification of analgesia and hyperalgesia, respectively. The need for two different stimuli was due to direction of the anticipated response. Specifically, to quantify analgesia an increase in withdrawal latency was required. Therefore, short baseline responses were needed to enable 7-8 s test range before the cut-off was achieved. For hyperalgesia, a decrease in withdrawal latency needed to be quantified. Therefore, a longer baseline latency was needed to be able to detect the decrease above the bottom limit of reflex tail withdrawal. The order of paw and tail testing varied randomly. Nociceptive assessments for acute administration experiments were then made at 0 (immediately following remote drug delivery), 5 min, 15 min and every 10 min thereafter until completion of the experiment only using the short baseline latency Hargreaves stimuli. For the chronic drug delivery experiments short and long baseline latency Hargreaves stimuli were employed on alternating time points, and in these experiments the rats were tested pre dose and 2 h post dose on days 1, 4 and 7 of dosing. For twice daily injections, the nociceptive response to the morning dose was assessed.

### Von Frey Test for Mechanical Allodynia.

Rats received at least three 60 min habituations to the test environment prior to behavioral testing. Response thresholds to calibrated light pressure stimuli applied to the plantar surface of the paws was measured using the von Frey test. The test was performed using 0.406-15.136 gm calibrated Semmes-Weinstein monofilaments (von Frey hairs; Stoelting, Wood Dale, Ill., USA) as described in detail previously (Brain Research 2000 861 105-116). Briefly, rats were first assessed for baseline response thresholds (average of three consecutive withdrawal assessments) from each paw at 15 min intervals, and the average response threshold from both feet was calculated. All testing was conducted blind with respect to group assignment. The behavioral responses were used to calculate the threshold, by fitting a Gaussian integral psychometric function using a maximum-likelihood fitting method (Percept. Psychophys. 1999 61 87-106; Behav. Res. Methods Instrum Comput. 1986 18 623-632), as described in detail previously (Brain Research 2000 861 105-116). Allodynia was assessed pre and post drug delivery.

### TIRAP Inhibitor Data

Rats had intrathecal catheters implanted using method 1. About 1 μl of 50 μM solution of TRAP inhibitor or control peptide (Imgenex, San Diego, Calif.) was administered at time of intrathecal catheter implant 2 hr before administration of 15 μg of morphine. Hargreaves testing was conducted as described above. As shown in FIG. 5, inhibition of TRAP signaling significantly potentiated intrathecal morphine analgesia.

### LPS Antagonist Data

Rats had intrathecal catheters implanted using method 1. About 20 μg of LPS antagonist in 4 μl (LPS from msbB E. coli mutant, which is a TLR4 antagonist due to this mutant LPS's lack of the myristoyl fatty acid moiety of lipid A, Invivogen, San Diego, Calif.) was co-administered intrathecally with morphine using a slightly modified catheter implantation protocol. Catheters were inserted only 7.7 cm rather than 8.8 cm due to the lack of diffusion within the intrathecal space by the LPS antagonist. As shown in FIG. 6, the LPS antagonist (and therefore TLR4 antagonist) significantly potentiated morphine analgesia.

### (+)-Naloxone & (+)-Naltrexone Plus Morphine Data

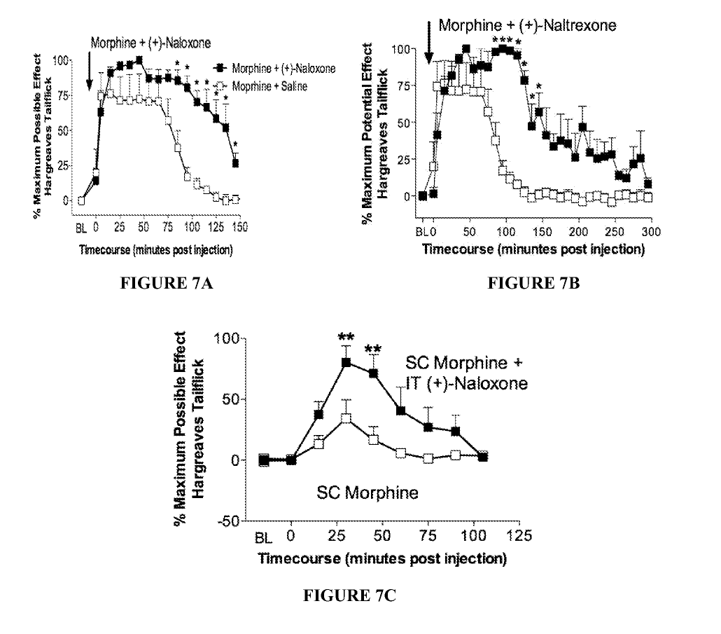

Rats had intrathecal catheters implanted using method 1. About 20 μg of (+)-naloxone and about 20 μg of (+)-naltrexone were co-administered with morphine intrathecally resulting in a significant potentiation of analgesia. See FIGS. 7A and 7B, respectively.

Rats were implanted with catheters using method 1 & 3. Morphine was delivered via the subcutaneous catheter (4 mg/kg). (+)-Naloxone was co-administered intrathecally (20 μg). Intrathecal co-administration with (+)-naloxone significantly potentiated morphine analgesia. See FIG. 7C.

### Chronic Systemic Morphine & (+)-Naloxone Experiment

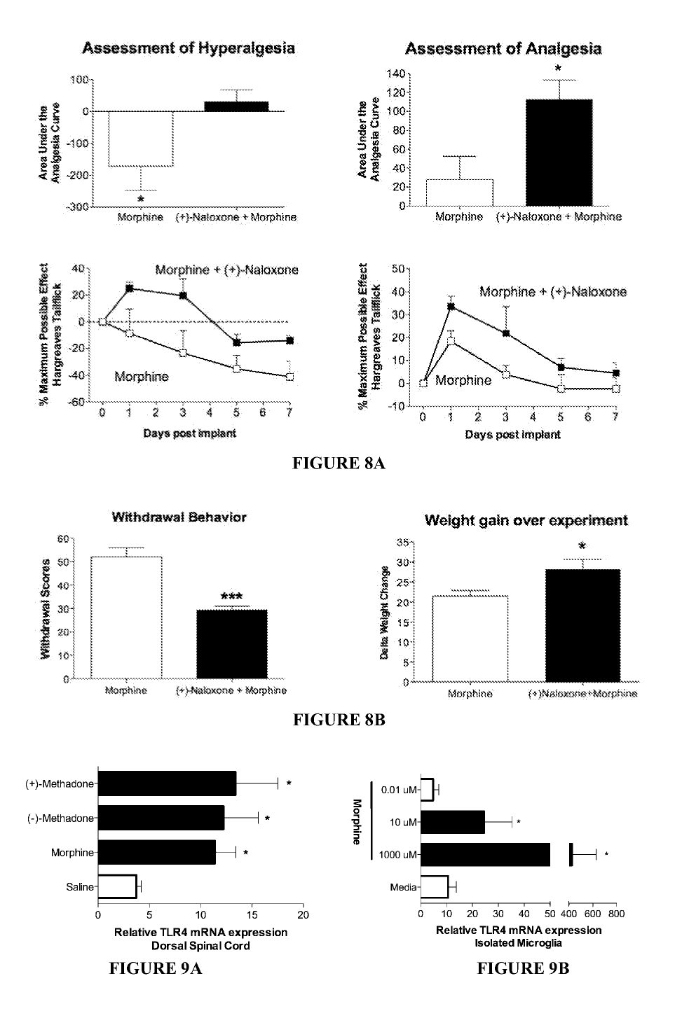

Rats were baselined on Hargreaves short latency (analgesia) and long latency (hyperalgesia) prior to surgery and then were subcutaneously implanted with 2 osmotic minipumps. One delivered 6 mg/day morphine and the other 12 mg/day (+)-naloxone or saline. Rats were tested on Hargreaves on days 1, 3, 5, and 7 after the pump implants. See FIG. 8A. After nociceptive testing on day 7 the animals received subcutaneous administration of 100 mg/kg (−)-naltrexone to precipitate opioid withdrawal. Withdrawal behaviors were then scored by 3 independent observers who were blinded to treatment groups. See FIG. 8B. Withdrawal behaviors that were scored included: jumping, rearing, exploration (movement greater than one body length), teeth chattering, wet dog shakes, abnormal posture, escape attempts, ptosis, diarrhea, penis licking, oral stimulation (filling of mouth with bedding), paw chewing, cleaning, salivation, vocalization, chewing (large jaw movements including masseter muscle contraction) and fidgeting (a writhing type of behavior involving small shifts in body position). Counts of each of these behaviors were made upon their presentation. In cases where the response was prolonged, for example ptosis, counts were made every 30 sec. Rat weight was recorded at time of surgery (post pump implant) and prior to withdrawal experiment. (+)-Naloxone co-administration potentiated morphine analgesia, attenuated the development of tolerance, blocked the development of morphine-induced hyperalgesia, reduced withdrawal behaviors and enabled normal weight gain in rats.

Modulation of TLR4 mRNA Expression by Opioids

Rats had intrathecal catheters implanted using a modified method 1. Rats received once daily for 7 days intrathecal morphine (15 μg), (+)-methadone (15 μg), (−)-methadone (15 μg) or saline. On day 7 the lumbar dorsal spinal cords were collected after transcardial saline perfusion. One hemisphere of the dorsal spinal cord was then used for mRNA quantification (the other hemisphere was used for protein). The samples were prepared for real-time reverse transcription-PCR cytokine mRNA quantification. They were expressed relative to the levels of the housekeeping gene GAPDH (M17701; forward: GTTTGTGATGGGTGTGAACC; reverse: TCTTCTGAGTGGCAGTGATG); Toll-like receptor 4 (NM_{—}019178; forward: CAGAGGAAGAACAAGAAGC; reverse: CCAGATGAACTGTAGCATTC). Primers were purchased from Proligo (Boulder, Colo., USA).

Chronic intrathecal opioid administration caused a significant elevation in TLR4 mRNA in a non-classical opioid fashion as both the opioid active (−)-methadone and the opioid inactive (+)-methadone caused the same elevation. See FIG. 9A.

The dorsal spinal cords of untreated rats were isolated and the microglia from this tissue were purified. Microglial cells were incubated with morphine in vitro for 2 hr. Following incubation, cells were collected and mRNA prepared for quantification. Morphine dose dependently increased isolated microglial TLR4 mRNA expression. See FIG. 9B. These data demonstrate that morphine is capable of causing direct microglial activation in vitro in the absence of any neuronal input.

### Reversal of Chronic Constriction Allodynia

Following baseline Von Frey testing described above, neuropathic pain was induced using the chronic constriction injury model of partial sciatic nerve injury. CCI was performed at mid-thigh level of the left hindleg. In brief, four sterile chromic gut sutures (cuticular 4-0 chromic gut, FS-2; Ethicon, Somerville, N.J.) were loosely tied around the gently isolated sciatic nerve, in the same surgery as for intrathecal catheter placements (above). Behavioral Von Frey testing was conducted on days 4 and 10 following CCI surgery to verify the development of exaggerated pain.

### Acute Reversal

Following behavioral testing on day 10 rats received acute lumbar puncture delivering 20 μg LPS antagonist or saline following which testing was contacted 1 and 4 hrs later.

### Chronic Reversal

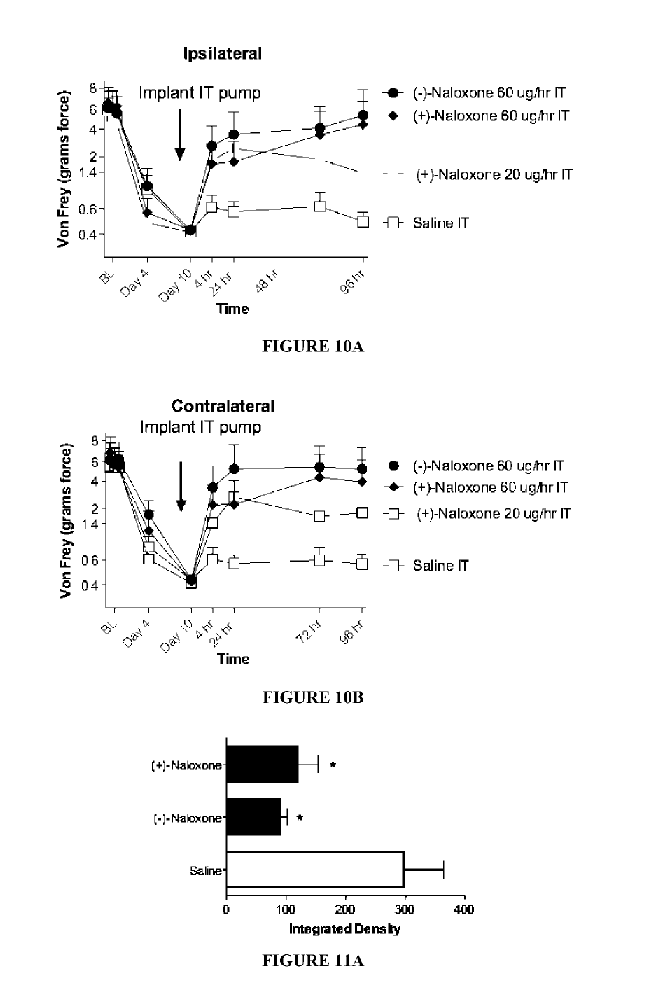

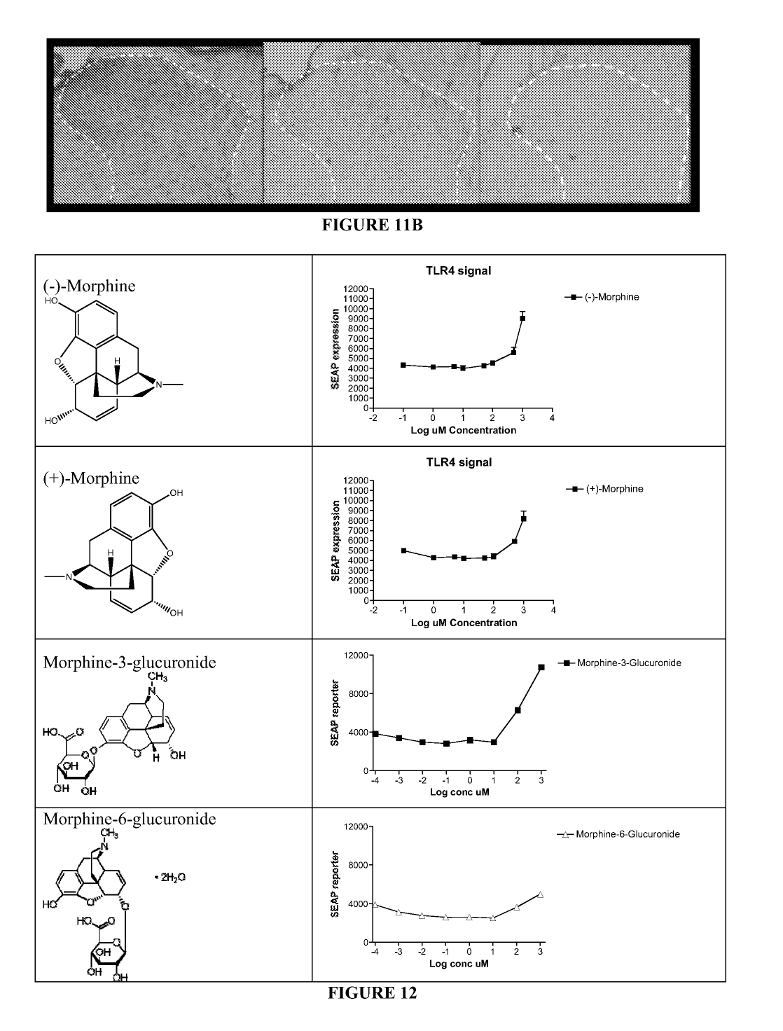

On day 10 following behavioral testing chronic indwelling intrathecal catheters were implanted using method 3, with osmotic minipumps delivering saline, 20 ug/hr (+)-naloxone, 60 ug/hr (+)-naloxone or 60 ug/hr (−)-naloxone. Von Frey testing was then conducted 4, 24, 72, and 96 hr post pump implant by testers blinded to treatment conditions. See FIGS. 10A and 10B.

Following the final behavioral testing rats were transcardially perfused first with isotonic saline and then with fresh 4% paraformaldehyde/0.1 M phosphate buffer (4% PFA/PB, pH 7.4). After dissection and immersion post-fixation in 4% PFA/PB for an additional 24 hr, the lumbar spinal cords then dehydrated in 30% sucrose with 0.1% azide at 4° C. until slicing.

For immunohistochemistry, sections were treated to suppress endogenous peroxidase and to prevent staining of endogenous biotin. The sections were incubated in either primary mouse monoclonal anti-rat glial fibrillary acidic protein (GFAP) (1:500; to visualize astrocyte astrocyte activation; ICN Biomedicals, Costa Mesa, Calif.) or primary mouse monoclonal anti-rat OX-42 (1:500; to visualize microglial activation; BioSource, Camarillo, Calif.) for 24 hr at 4° C. The slides were then incubated in secondary biotinylated goat anti-mouse IgG antibody (1:500; Jackson ImmunoResearch, West Grove, Pa.) overnight at 4° C. Finally sections were reacted using the avidin-biotin complex procedure (ABC, Vector Elite kit; 1:100 in PBS-Triton; 2 hr; Vector laboratories, Burlingame, Calif.) and 3′,3′-diaminobenzidine (DAB; Sigma-Aldrich). Glucose oxidase (Sigma-Aldrich; type V-s; 0.02%) and beta-D-glucose (0.1%) were used to generate hydrogen peroxide. Nickel (I) ammonium sulfate was added to the DAB solution (0.025% w/v) to intensify the reaction product. One series of sections was processed as described above except that the primary antibody was omitted from the incubation buffer (omission controls). The slides were dried overnight, cleared, and cover slipped.

Slides were viewed with an Olympus Vannox II bright-field microscope. Images were collected with a Cohu CCD camera coupled to an Apple PowerMac 7200 equipped with NIH Image software (version 1.60). Brightness and contrast were kept constant with the images not altered. The intensity of the light source was calibrated each day.

Quantitative integrated density (FIG. 11A) and example microphotographs of CD11b (OX42, FIG. 11B) staining in the left dorsal horn of rats with chronic constriction injury of the left sciatic nerve following chronic intrathecal treatment with saline, (+)-naloxone or (−)-naloxone.

These data demonstrate that TLR4 antagonism acutely and chronically reverses neuropathic pain & is associated with a reduction in microglial activation

### In Vitro TLR Cell Line Data

### Cell Culture and Reporter Protein Assay

A human embryonic kidney-293 (HEK 293) cell line stably transfected to express human TLR4 at high levels was purchased from Invivogen (293-htlr4a-md2cd14; here referred to as HEK-TLR4). These cells are stably transfected by Invivogen with multiple genes involved in TLR4 recognition that include TLR4 and the co-receptors MD2 and CD14. In addition, these cells stably express an optimized alkaline phosphatase reporter gene under the control of a promoter inducible by several transcription factors such as NF-kB and AP-1. Secreted alkaline phosphatase (SEAP) protein was produced as a consequence of TLR4 activation.

HEK-TLR4 cells were grown at 37° C. (5% CO₂; VWR incubator model 2300) in 10-cm dishes (Greiner Bio-One, CellStar 632171; Monroe, N.C.) in normal supplement selection media (DMEM media [Invitrogen, Carlsbad, Calif.] supplemented with 10% fetal bovine serum [Hyclone; Logan, Utah], HEK-TLR4 selection [Invivogen]; Penicillin 10,000U/ml [Invitrogen]; Streptomycin 10 mg/ml [Invitrogen], Normocine [Invivogen], and 200 nM L-Glutamin [Invitrogen]). The cells were then plated for 48 hr in 96 well plates (Microtest 96 well plate, flat bottom, Becton Dickinson; 5×10³ cells/well) with the same media. After 48 hr, supernatants were removed and replaced with 180-μl artificial cerebrospinal fluid (sterile aCSF; 124 mM NaCl, 5 mM KCl, 0.1 mM CaCl₂.2H₂O, 3.2 mM MgCl₂.6H₂O, 25 mM NaHCO₃, 10 mM glucose, pH 7.4) to model in vivo conditions. Drugs under test were then added in 20 μl and incubated for 24 hr. Supernatants (15 μl) were then collected from each well for immediate assay.

SEAP in the supernatants was assayed using the Phospha-Light System (Applied Biosystems) according to manufacturer's instructions. This is a chemiluminescent assay that incorporates Tropix CSPD chemiluminescent substrate. The 15 μl test samples were diluted in 45 μl of 1× dilution buffer, transferred to 96-well plates (Thermo, Walthma, Mass.), and heated at 65° C. in a water bath (Model 210, Fisher Scientific, Pittsburgh, Pa.) for 30 min, then cooled on ice to room temperature. Assay buffer (50 μl/well) was added and, 5 min later, reaction buffer (50 μl/well) is added and allowed to incubate for 20 min at room temperature. The light output was then measured in a microplate luminometer (Dynex Technologies, #IL213.1191, Chantilly, Va.).

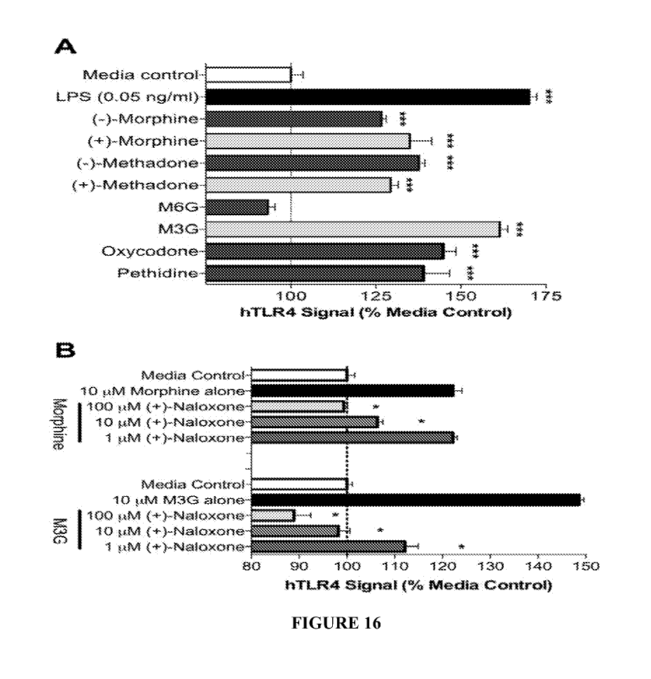

FIG. 16A shows a result of comparison experiment of the agonist (and lack of) activity of opioids on TLR4 transfected HEK293 cells compared to LPS. FIG. 16B shows attenuation of morphine & M3G induced TLR4 signaling in the TLR4 transfected HEK293 cells by a variety of doses of (+)-naloxone.

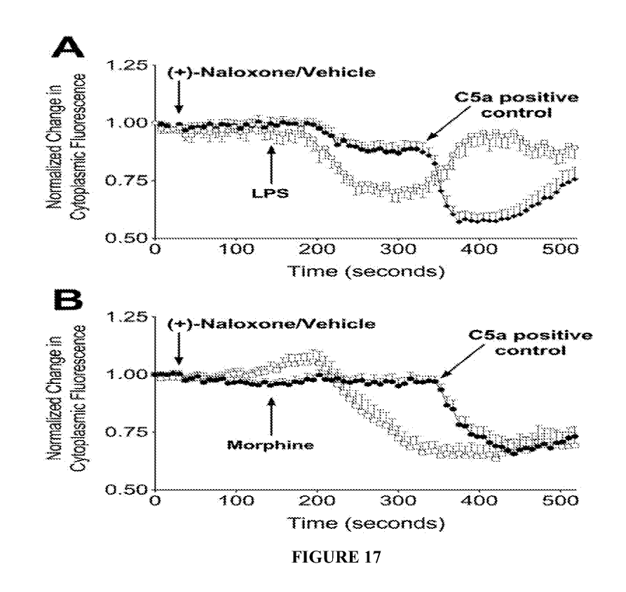

FIG. 17A shows results of experiments conducted using a RAW264.7 mouse macrophage cell line that stably expresses green fluorescent protein (GFP)-tagged Akt1. Under basal conditions, Akt1 is diffusely distributed in the cytosol but, upon activation, rapidly moves to the membrane site where an Akt1 activating event is occurring. In the case of LPS-induced TLR4 signaling, activated Akt1 moves out of the cytosol to the TLR4 lipid raft. As expected, LPS (7.5 μg/mL in 1.2 mL total dish volume) reliably induced robust, rapid clearance of GFP-Akt1 from the cytosol resulting from LPS-TLR4 induced membrane localization of the GFP-Akt1 (open squares). In contrast, preincubation with either (+)-naloxone (1mM) prior to addition of LPS significantly attenuated the Akt1 response.

Using the same GFP-Akt1 RAW264.7 cell line as used for the LPS experiment, morphine (100 μM) was found to cause significant and prolonged clearance of GFP-Akt1 from the cytosol (FIG. 17B) resulting from membrane localization of the GFP-Akt1. This morphine-induced Akt1 response was blocked by (+)-naloxone, thereby implicating involvement of TLR4 receptor agonist activity rather than opioid receptor. To clarify whether the blockade of the Akt1 by naloxone response was specific to the TLR4 signaling pathway, rather than a general suppression of Akt1 itself, complement 5a (C5a; 25 ng/ml) was applied to the naloxone-TLR4 blocked cells, immediately precipitating significant Akt1 membrane localization quantified by cytosolic clearance (FIGS. 17A and B).

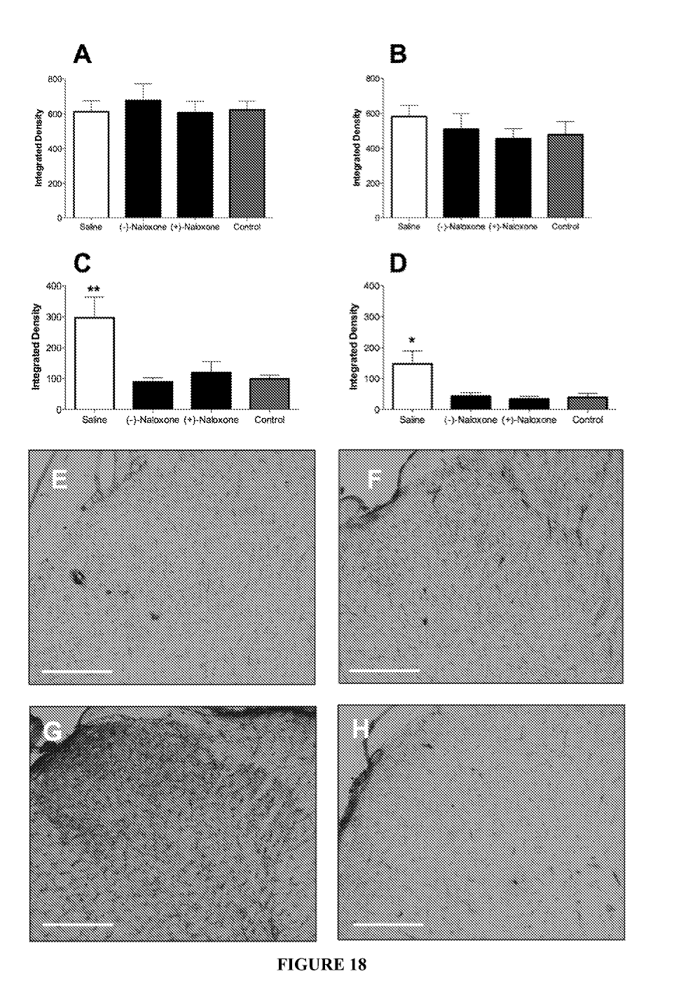

The L5 spinal cord of CCI rats receiving sustained intrathecal administration of 60 μg/hr (−)-naloxone, 60 μg/hr (+)-naloxone, or vehicle across 4 days were analyzed for expression of astrocyte (GFAP; FIGS. 18A and B) and microglia (CD11b/c; FIGS. 18C-H) activation markers. As quantified by densitometry, neither (+)-nor (−)-naloxone affected GFAP expression in ipsilateral (FIG. 18A) or contralateral (FIG. 18B) dorsal horns, compared to vehicle controls. In contrast, both isomers of naloxone produced reliable suppression of CD11b/c bilaterally, compared to neuropathic vehicle treated animals (FIGS. 18C and D). Representative sections (10×) of ipsilateral dorsal horns of neuropathic (CCI) rats treated intrathecally with 60 μg/hr (+)-naloxone (FIG. 11E), 60 μg/hr (−)-naloxone (FIG. 18F), and vehicle (FIG. 18G) are shown. Comparable section from a naïve control is illustrated in FIG. 18H. *=p<0.05, **=p<0.01 compared to naive controls. Scale bar=500 μm.

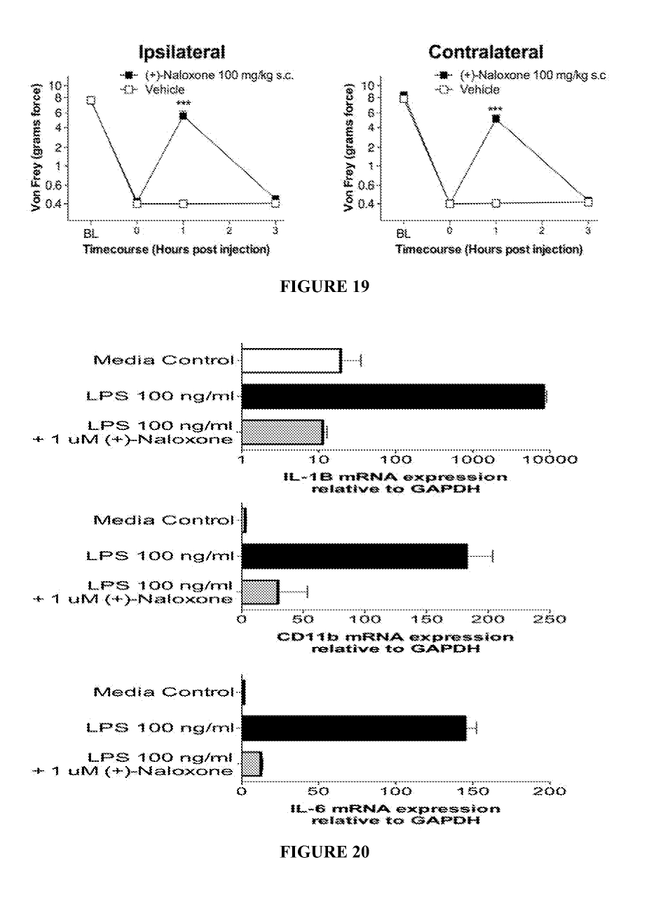

After baseline (BL) testing, rats received CCI of one sciatic nerve at mid-thigh level. After pre-drug testing (0 hr) 14 days later to confirm the development of bilateral CCI-induced mechanical allodynia, rats were administered 100 mg/kg subcutaneous (+)-naloxone (filled squares in FIG. 19) or vehicle (open symbols in FIG. 19). As shown in FIG. 19, behavioral responses recorded after drugs revealed reliable attenuation of both ipsilateral and contralateral mechanical allodynia by systemic (+)-naloxone. ***=p<0.001 compared to vehicle (saline) controls.

HAPI microglial cell line was used to examine the effects of (+)-naloxone on microglial activation by LPS. Whereas 100 ng/mL LPS produced elevations in mRNA for CD11b (microglial activation marker), interleukin (IL)-1b, and IL-6, 1 μM (+)-naloxone abolished each of these LPS-induced effects. See FIG. 20 (GAPDH=glyceraldehyde-3-phosphate-dehydrogenase).

### Activity of TLR Agonists and Antagonists

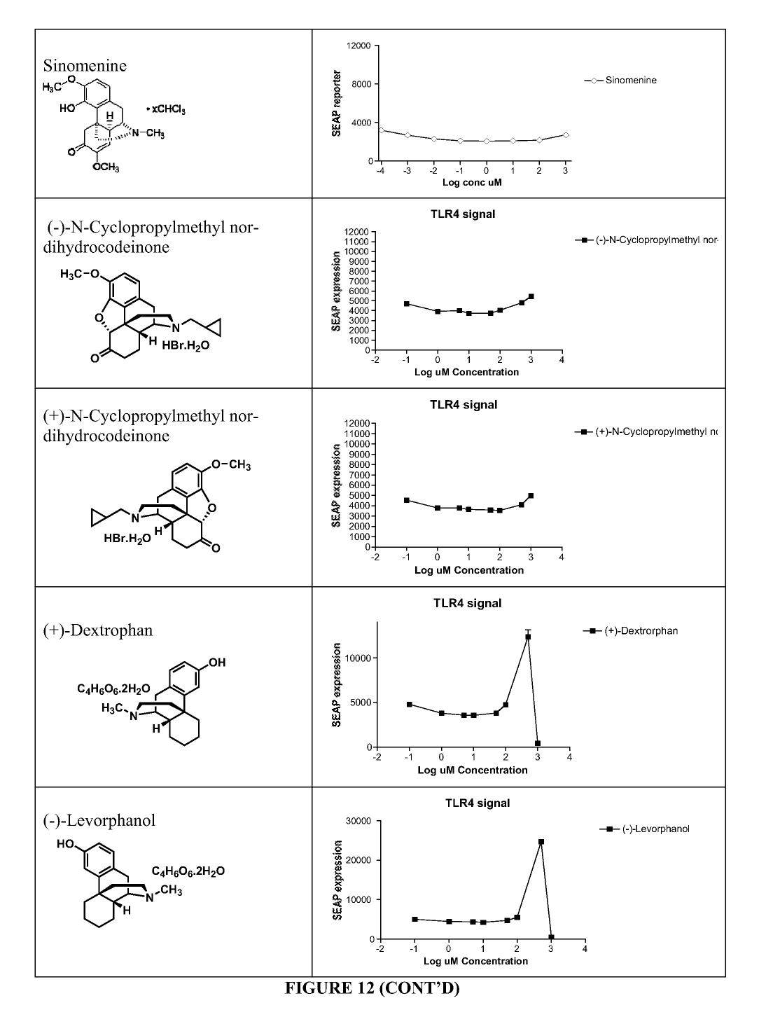

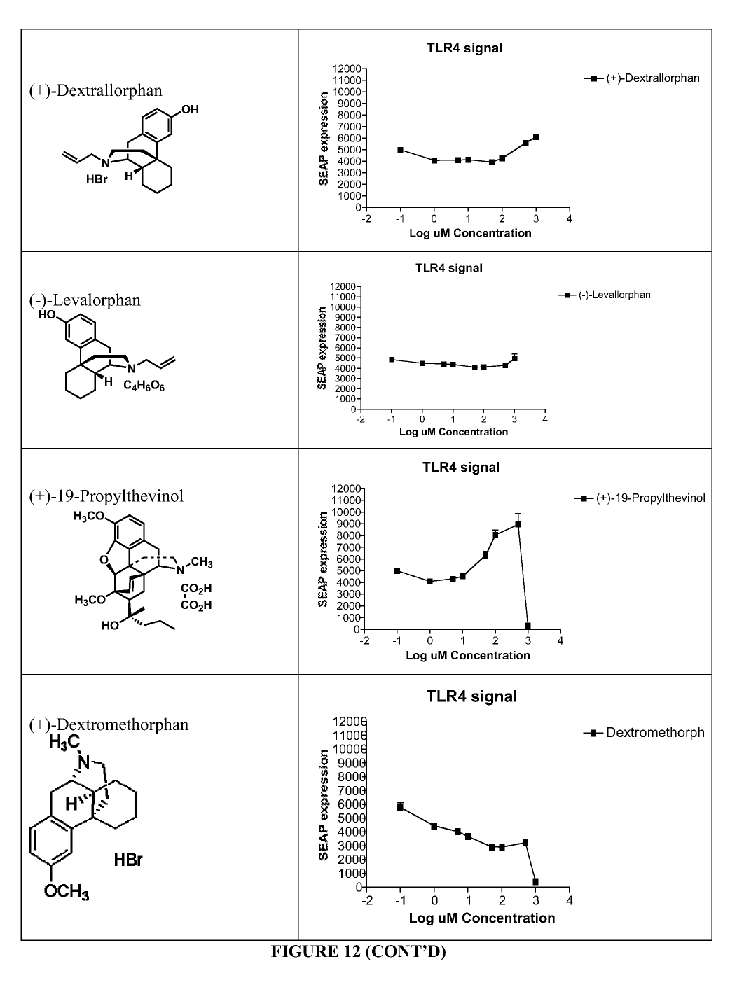

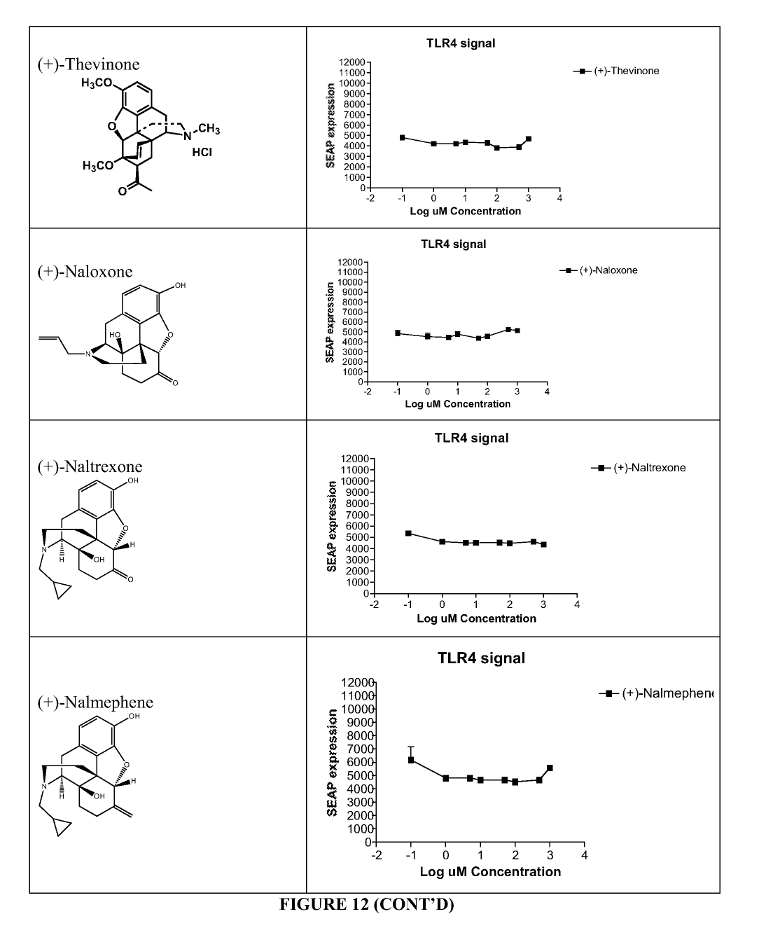

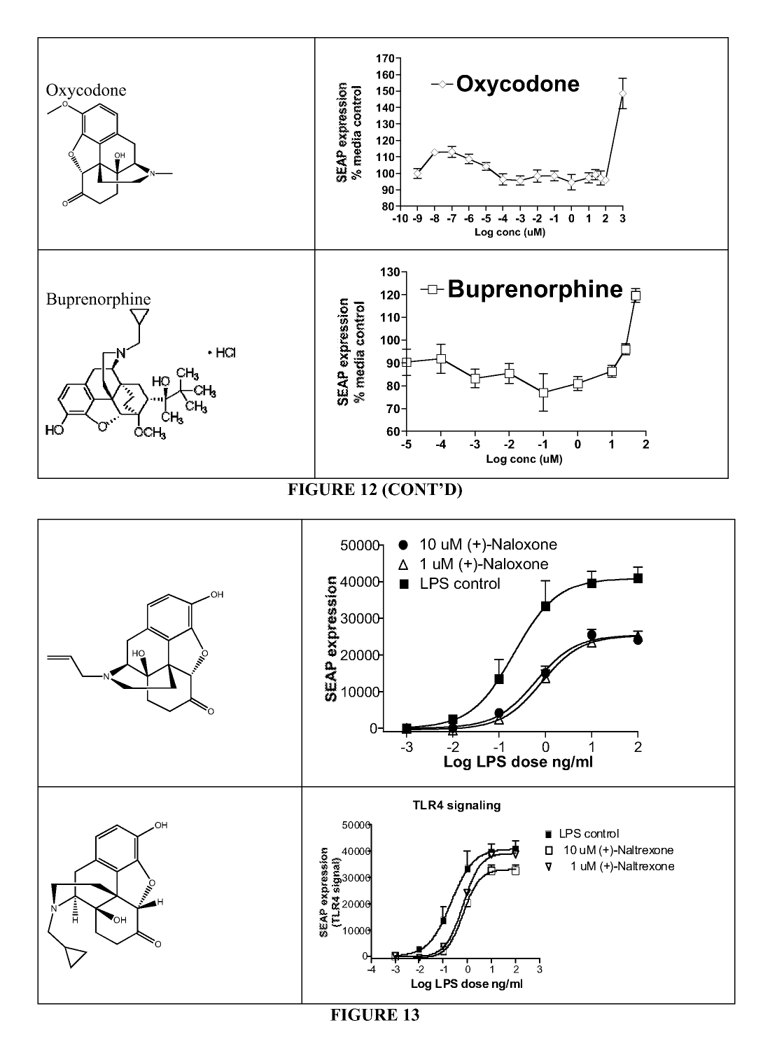

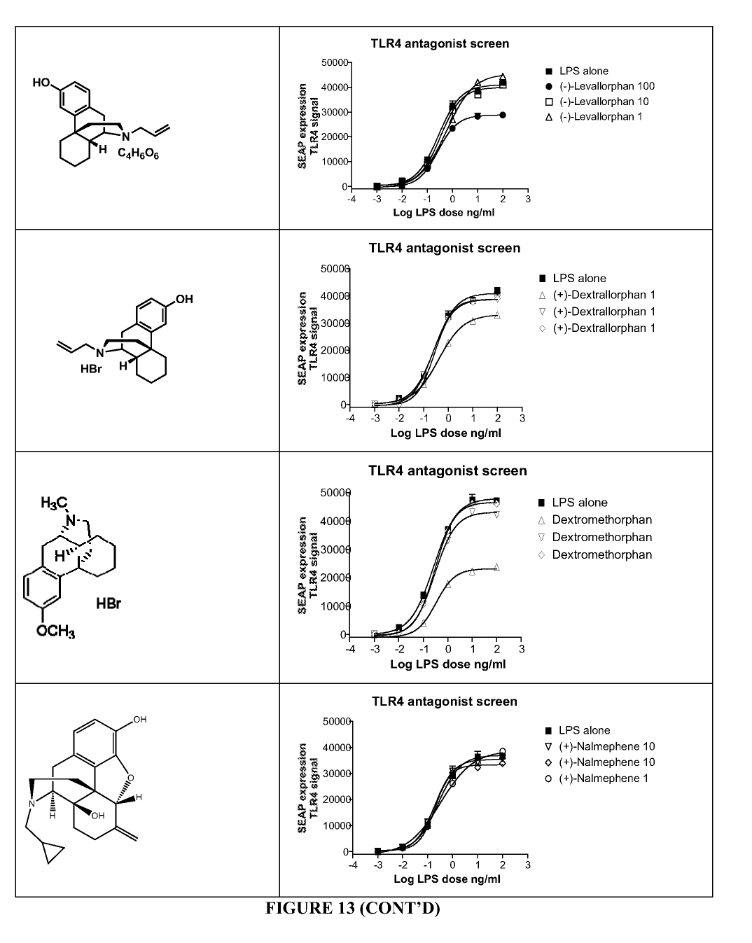

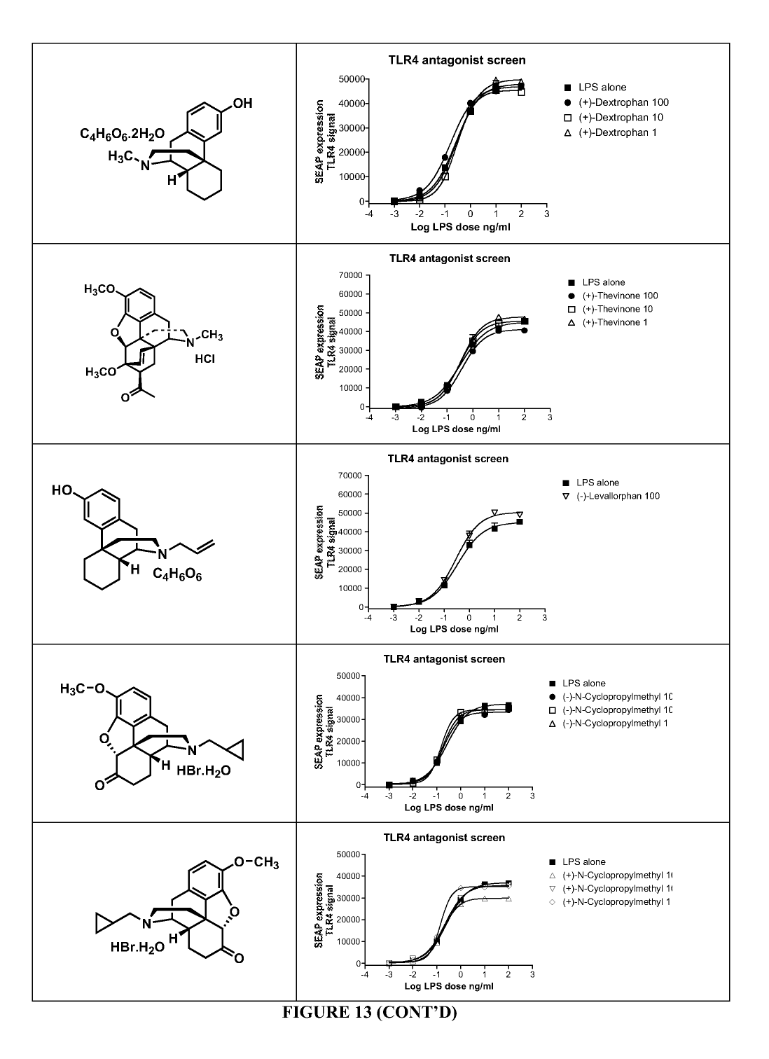

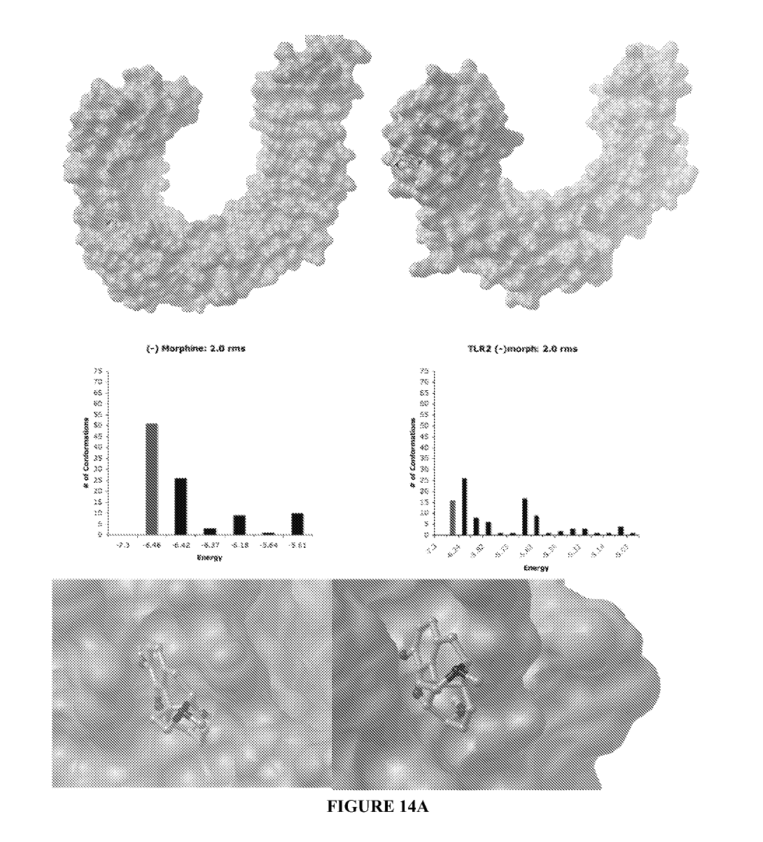

Some of the opioids and the corresponding TLR agonist activity are shown in FIG. 12. In addition, some of the opioids and the corresponding TLR antagonistic activity are shown in FIG. 13.

### Drugs

(−)-Morphine sulfate was obtained from Mallinckrodt, Inc. (St. Louis, Mo.). (+)-Morphine sulfate, (+)-methadone, (−)-methadone, oxycodone, fentanyl, meperidine (+)-naloxone, (−)-naloxone, (+)-naltrexone and (−)-naltrexone were obtained from the National Institute on Drug Abuse (Research Triangle Park, N.C., USA). Other opioids were purchased from Sigma. Sterile endotoxin-free isotonic saline (Abbott Laboratories, North Chicago, Ill.) was the vehicle for all opioids. Lipopolysaccharide (LPS; a classical TLR4 agonist) was purchased from Sigma (St. Louis, Mo.) and LPS from msbB E. coli mutant, which is a TLR4 antagonist due to this mutant LPS's lack of the myristoyl fatty acid moiety of lipid A, was purchased from Invivogen (San Diego, Calif.). Interleukin-1 receptor antagonist (IL-1ra) was obtained from Amgen (Thousand Oaks, Calif.). Where applicable, drugs were prepared and are reported as free base concentrations. Vehicles were administered equavolume to the drugs under test.

### Computational Modeling

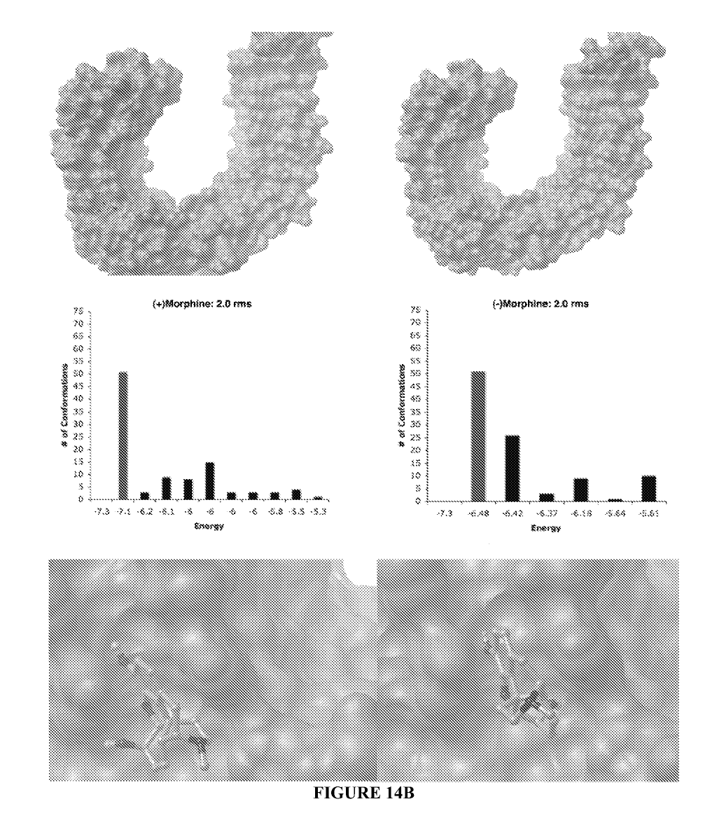

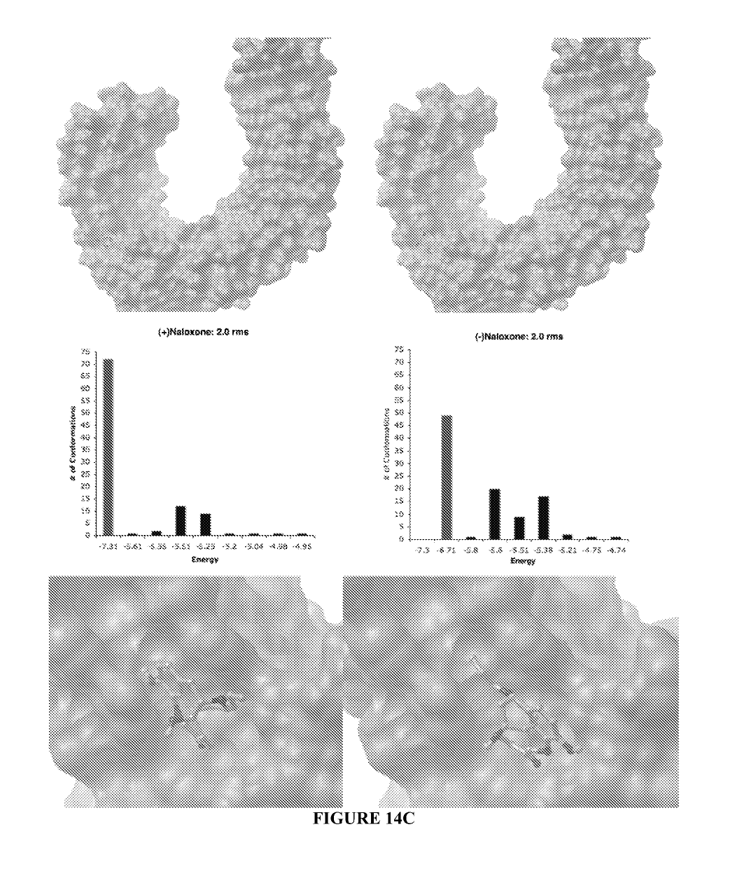

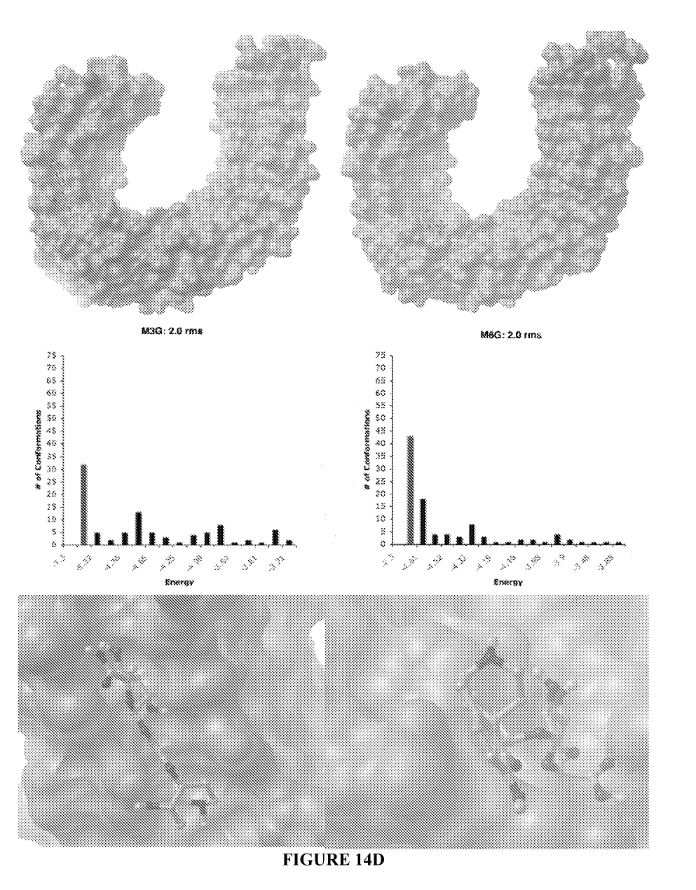

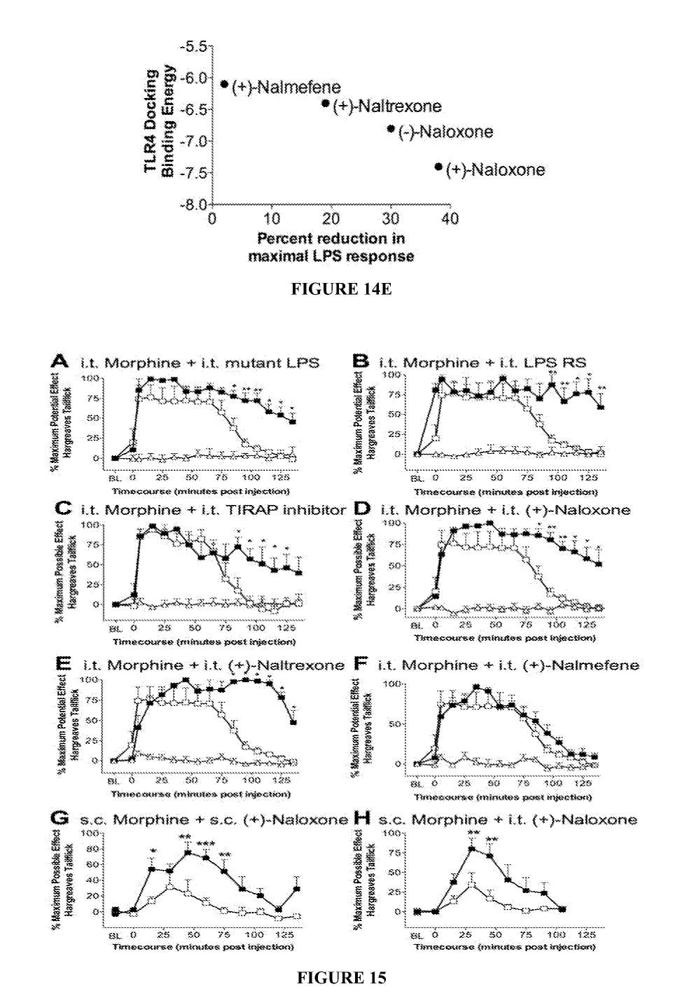

Suitable TLR antagonists can be obtained by computer modeling. FIG. 14A shows docking simulations of (−)-Morphine in silico TLR4 & TLR2. The TLR4 pdb file was obtained from RCSB database (ID 2z64) with binding partner MD-2, which was removed as were all ligands via Molegro Molecular Viewer. RCSB also produced TLR2 (ID 2z80) as a heterodimer with TLR1, which was eliminated with same methods. Modified pdb files were input into AutoDock 4.0, hydrogens added and resaved in pdbqt format. Drug ligands for docking were gathered using PubChem isomeric SMILES then converted to .pdb using a structure file generator (http://cactus.nci.nih.gov/services/translate/). Ligand torsion tree roots were generated automatically by choosing ‘detect root’. Figures shown cover lower energy results from left portion of receptor only, with AutoGrid center set at (−44.725, 4.064, −0.446) for TLR4; (−31.851, 34.759, 5.638) for TLR2, all with 126 grid points expanding each direction. The search parameters employed a genetic algorithm set for #GA 100, Max Evaluations 5×10⁶ and 0.375 Å spacing. All dockings executed with Lamarkian genetic algorithms on Apple desktop computers running OS×10.4.

As shown in FIG. 14A, (−)-morphine docked to different pockets on TLR2 & TLR4, with TLR4 docking showing greater selectivity to one pocket with the greatest frequency & lowest energy requirements for this conformation.

FIG. 14B is in silico TLR4 docking simulations of (−)- and (+)-morphine using the methods described above. As can be seen in FIG. 14B, (+)- and (−)-morphine dock to the same pocket on TLR4, albeit in different conformations.

FIG. 14C is in silico TLR4 docking simulation of (+)- and (−)-naloxone. using the methods described above. As can be seen, naloxone docks non-stereoselectively to the same pocket that morphine non-stereoselectively binds to.

FIG. 14D is in silico TLR4 docking simulation of M3G and M6G. As can be seen, M3G appears to dock to the same location as morphine and naloxone, whereas M6G did not show the same preference for this pocket, thereby indicating different activity. In vitro assay confirmed different activity as M6G was not found to have any significant TLR4 agonist activity while M3G showed TLR4 agonist activity.

FIG. 14E is a graph of in silico docking energies (y-axis) vs. in vitro % reduction in LPS response (x-axis) of various compounds. FIG. 14E provides at least some validation of the in silico docking simulation model.

### Potentiation of Opioid Analgesia

Rats had intrathecal catheters implanted using method 1. About 20 μg of various TLR antagonists was co-administered with morphine intrathecally resulting in a significant potentiation of analgesia. See FIGS. 15A-H, where white square represents morphine alone, black square represents morphine+TLR antagonist, and white triangle represents control (i.e., only vehicle).

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed:

1. A composition comprising an admixture of a therapeutically effective amount of an analgesic opioid and a TLR antagonist, wherein said TLR antagonist comprises 4,5-epoxymorphinan or a derivative thereof, a morphinan or a derivative thereof, or a pharmaceutical salt or a prodrug thereof.

2. The composition of claim 1, wherein said TLR antagonist is of the formula: 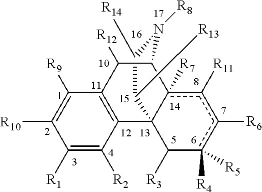
wherein
each of R¹, R⁹, R¹⁰, R¹¹, R¹² is independently H, alkyl, —XR¹⁵, halide, —NR¹⁶R¹⁷, carboxyl, hydroxyalkyl, haloalkyl, cycloalkyl, (cycloalkyl)alkyl, acyl, alkoxyalkyl, alkoxycarbonyl, alkyl-carboxyl, aryl, aralkyl, aryloxy, arylthio, alkylthio, or amido;
R² is hydrogen, alkyl, halide, OR′, where R′ is hydrogen, hydroxyalkyl, haloalkyl, cycloalkyl, (cycloalkyl)alkyl, alkoxyalkyl, alkenyl, aryl, aralkyl, aryloxy, alkylthio, amino, amido, carboxyl, or —C(OR^{b})R^{c}R^{d}, wherein each of R^{b}, R^{c}, and R^{d} is independently H, alkyl, cycloalkyl, phenyl, or phenalkyl;
R³ is hydrogen, alkyl, or halide;
or R² and R³ together form —NR^{a}—, —O—, or —S—, where R^{a} is hydrogen, alkyl, or a nitrogen protection group;
R⁴ is H, —XR¹⁵, or —OC(═O)—R¹⁸;
R⁵ is H;
or R⁴ and R⁵ together form ═Y;
R⁶ is H, —XR¹⁹, alkyl, —C(═O)—R¹⁸, hydroxyalkyl, haloalkyl, cycloalkyl, (cycloalkyl)alkyl, alkoxyalkyl, alkenylalkyl, aryl, aralkyl, aryloxy, alkylthio, amino, amido, carboxyl, or —C(OR^{b})R^{c}R^{d}, wherein each of R^{b}, R^{c}, and R^{d} is independently H, alkyl, cycloalkyl, phenyl, or phenalkyl;
R⁷ is H, alkyl, or —XR¹⁵;
or R⁵ and R⁷ together form —(CH₂)ₙ—;
R⁸ is H, alkyl, haloalkyl, a nitrogen protecting group, (cycloalkyl)alkyl, or alkenyl;
each of R¹³ and R¹⁴ is independently halide, oxime, alkyl, arylalkyl, CN, —OR¹⁸ and —OC(═O)—R¹⁸;
each R¹⁵ is independently H, alkyl, hydroxyalkyl, haloalkyl, alkoxyalkyl, haloacyl, acyl, aryl, aralkyl, or carbohydrate or a derivative thereof;
R¹⁶ is hydrogen, alkyl, or a nitrogen protecting group;
each of R¹⁷, R¹⁸, and R¹⁹ is independently hydrogen or alkyl;
n is an integer from 1 to 4;
each X is independently O or S;
Y is O or CH₂; and
dashed line indicates an optional double bond provided that when C₇-C₈ is a double bond, C₆-C₇ and C₈-C₁₄ are single bonds, and provided that when C₈-C₁₄ is a double bond R⁷ is absent.

3. The composition of claim 2, wherein R¹ is —OR¹⁵, wherein R¹⁵ is hydrogen, alkyl, or a carbohydrate or a derivative thereof.

4. The composition of claim 2, wherein
R⁴ is —OR¹⁵, or —OC(═O)—R¹⁹, and R⁴ is H, wherein R¹⁵ is hydrogen, alkyl, or a carbohydrate or a derivative thereof, and R¹⁹ is alkyl;
or R⁴ and R⁵ together form ═O or ═CH₂.

5. The composition of claim 2, wherein
R⁷ is H, —OR¹⁵, and R¹⁵ is hydrogen or alkyl;
or R⁵ and R⁷ together form —(CH₂)₂—.

6. The composition of claim 2, wherein R⁸ is alkyl, haloalkyl, (cycloalkyl)alkyl, or alkenyl.

7. The composition of claim 2, wherein R⁶ is hydrogen or hydroxyalkyl.

8. The composition of claim 2, wherein X is O.

9. The composition of claim 2, wherein C₇-C₈ is a double bond, and C₆-C₇ and C₈-C₁₄ are single bonds.

10. The composition of claim 2, wherein C₇-C₈ is a single bond, and C₆-C₇ and C₈-C₁₄ are double bonds and R⁷ is absent.

11. The composition of claim 1, wherein said TLR antagonist is not an enantiomer of said analgesic opioid.

12. The composition of claim 1, wherein said TLR antagonist is an enantiomer of said analgesic opioid.

13. A method of treating a subject for a clinical condition associated with Toll-like receptor (TLR) activation, said method comprising administering to the subject an effective amount of a TLR antagonist comprising 4,5-epoxymorphinan or a derivative thereof, a morphinan or a derivative thereof, or a pharmaceutical salt or a prodrug thereof.

14. The method of claim 13, wherein the clinical condition comprises a condition associated with Toll-like receptor (TLR) mediated activation of glial, cell, or a combination thereof.

15. The method of claim 13, wherein the clinical condition associated with TLR activation comprises chronic pain, acute opioid analgesia, or an opioid effect that occurs as a consequence of TLR activation, gastrointestinal pathologies, cardiovascular disease, diabetes, immune related conditions, systemic pathologies, neurodegeneration, induction of labor, fever, seizures, epilepsy, epileptogenesis, nociception, or a combination thereof.

16. The method of claim 15, wherein the clinical condition associated with TLR activation comprises chronic pain, nociception, acute opioid analgesia, or an opioid effect that occurs as a consequence of TLR activation, or a combination thereof.

17. The method of claim 13, wherein the TLR antagonist is a compound of the formula: 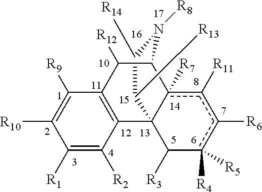
wherein
each of R¹, R⁹, R¹⁰, R¹¹, R¹² is independently H, alkyl, —XR¹⁵, halide, —NR¹⁶R¹⁷carboxyl, hydroxyalkyl, haloalkyl, cycloalkyl, (cycloalkyl)alkyl, acyl, alkoxyalkyl, alkoxycarbonyl, alkyl-carboxyl, aryl, aralkyl, aryloxy, arylthio, alkylthio, or amido;
R² is hydrogen, alkyl, halide, OR′, where R′ is hydrogen, hydroxyalkyl, haloalkyl, cycloalkyl, (cycloalkyl)alkyl, alkoxyalkyl, alkenyl, aryl, aralkyl, aryloxy, alkylthio, amino, amido, carboxyl, or —C(OR^{b})R^{c}R^{d}, wherein each of R^{b}, R^{c}, and R^{d} is independently H, alkyl, cycloalkyl, phenyl, or phenalkyl;
R³ is hydrogen, alkyl, or halide;
or R² and R³ together form —NR^{a}—, —O—, or —S—, where R^{a} is hydrogen, alkyl, or a nitrogen protection group;
R⁴ is H, —XR¹⁵, or —OC(═O)—R¹⁸;
R⁵ is H;
or R⁴ and R⁵ together form ═Y;
R⁶ is H, —XR¹⁹, alkyl, —C(═O)—R¹⁸, hydroxyalkyl, haloalkyl, cycloalkyl, (cycloalkyl)alkyl, alkoxyalkyl, alkenylalkyl, aryl, aralkyl, aryloxy, alkylthio, amino, amido, carboxyl, or —C(OR^{b})R^{c}R^{d}, wherein each of R^{b}, R^{c}, and R^{d} is independently H, alkyl, cycloalkyl, phenyl, or phenalkyl;
R⁷ is H, alkyl, or —XR¹⁵;
or R⁵ and R⁷ together form —(CH₂)ₙ—;
R⁸ is H, alkyl, haloalkyl, a nitrogen protecting group, (cycloalkyl)alkyl, or alkenyl;
each of R¹³ and R¹⁴ is independently halide, oxime, alkyl, arylalkyl, CN, —OR¹⁸ and —OC(═O)—R¹⁸;
each R¹⁵ is independently H, alkyl, hydroxyalkyl, haloalkyl, alkoxyalkyl, haloacyl, acyl, aryl, aralkyl, or carbohydrate or a derivative thereof;
R¹⁶ is hydrogen, alkyl, or a nitrogen protecting group;
each of R¹⁷, R¹⁸, and R¹⁹ is independently hydrogen or alkyl;
n is an integer from 1 to 4;
each X is independently O or S;
Y is O or CH₂; and
dashed line indicates an optional double bond provided that when C₇-C₈ is a double bond, C₆-C₇ and C₈-C₁₄ are single bonds, and provided that when C₈-C₁₄ is a double bond R⁷ is absent.

18. A method for potentiating analgesic effects of an analgesic opioid in a subject, said method comprising co-administering to the subject who is in need of an opioid treatment an effective amount of (i) an analgesic opioid and (ii) a TLR antagonist comprising a (+)-4,5-epoxymorphinan or a derivative thereof, a (+)-morphinan or a derivative thereof, or a pharmaceutical salt or a prodrug thereof.

19. The method of claim 18, wherein the TLR antagonist is a compound of the formula: 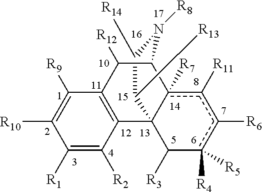
wherein
each of R¹, R⁹, R¹⁰, R¹¹, R¹² is independently H, alkyl, —XR¹⁵, halide, —NR¹⁶R¹⁷, carboxyl, hydroxyalkyl, haloalkyl, cycloalkyl, (cycloalkyl)alkyl, acyl, alkoxyalkyl, alkoxycarbonyl, alkyl-carboxyl, aryl, aralkyl, aryloxy, arylthio, alkylthio, or amido;
R² is hydrogen, alkyl, halide, OR′, where R′ is hydrogen, hydroxyalkyl, haloalkyl, cycloalkyl, (cycloalkyl)alkyl, alkoxyalkyl, alkenyl, aryl, aralkyl, aryloxy, alkylthio, amino, amido, carboxyl, or —C(OR^{b})R^{c}R^{d}, wherein each of R^{b}, R^{c}, and R^{d} is independently H, alkyl, cycloalkyl, phenyl, or phenalkyl;
R³ is hydrogen, alkyl, or halide;
or R² and R³ together form —NR^{a}—, —O—, or —S—, where R^{a} is hydrogen, alkyl, or a nitrogen protection group;
R⁴ is H, —XR¹⁵, or —OC(═O)—R¹⁸;
R⁵ is H;
or R⁴ and R⁵ together form ═Y;
R⁶ is H, —XR¹⁹, alkyl, —C(═O)—R¹⁸, hydroxyalkyl, haloalkyl, cycloalkyl, (cycloalkyl)alkyl, alkoxyalkyl, alkenylalkyl, aryl, aralkyl, aryloxy, alkylthio, amino, amido, carboxyl, or —C(OR^{b})R^{c}R^{d}, wherein each of R^{b}, R^{c}, and R^{d} is independently H, alkyl, cycloalkyl, phenyl, or phenalkyl;
R⁷ is H, alkyl, or —XR¹⁵;
or R⁵ and R⁷ together form —(CH₂)ₙ—;
R⁸ is H, alkyl, haloalkyl, a nitrogen protecting group, (cycloalkyl)alkyl, or alkenyl;
each of R¹³ and R¹⁴ is independently halide, oxime, alkyl, arylalkyl, CN, —OR¹⁸ and —OC(═O)—R¹⁸;
each R¹⁵ is independently H, alkyl, hydroxyalkyl, haloalkyl, alkoxyalkyl, haloacyl, acyl, aryl, aralkyl, or carbohydrate or a derivative thereof;
R¹⁶ is hydrogen, alkyl, or a nitrogen protecting group;
each of R¹⁷, R¹⁸, and R¹⁹ is independently hydrogen or alkyl;
n is an integer from 1 to 4;
each X is independently O or S;
Y is O or CH₂; and
dashed line indicates an optional double bond provided that when C₇-C₈ is a double bond, C₆-C₇ and C₈-C₁₄ are single bonds, and provided that when C₈-C₁₄ is a double bond R⁷ is absent.

20. The method of claim 18, wherein said method also reduces the risk of developing an opioid dependency by a subject.


SEQUENCE LISTING (4 sequences)

SEQ ID NO: 1 - DNA, 20 bp, Artificial Sequence
  Forward Primer Sequence for GAPDH gene
gtttgtgatgggtgtgaacc

SEQ ID NO: 2 - DNA, 20 bp, Artificial Sequence
  Reverse Primer Sequence for GAPDH gene
tcttctgagtggcagtgatg

SEQ ID NO: 3 - DNA, 19 bp, Artificial Sequence
  Forward Primer Sequence for TLR-4
cagaggaagaacaagaagc

SEQ ID NO: 4 - DNA, 20 bp, Artificial Sequence
  Reverse Primer Sequence for TLR-4
ccagatgaactgtagcattc